(19) United States
(12) Patent Application Publication
(10) Pub. No.: US 2022/0118236 A1
(43) Pub. Date: Apr. 21, 2022

(54) ROLL-ON APPLICATORS AND METHODS FOR TREATING DERMAL CONDITIONS

(71) Applicant: Journey Medical Corporation, Scottsdale, AZ (US)

(72) Inventors: Sylvia YEP, Scottsdale, AZ (US); Carla VIDAL, Scottsdale, AZ (US); Fery PRANADI, Scottsdale, AZ (US); Mukund PATEL, Scottsdale, AZ (US); Delphine IMBERT, Scottsdale, AZ (US)

(21) Appl. No.: 17/506,343

(22) Filed: Oct. 20, 2021

Related U.S. Application Data
(60) Provisional application No. 63/094,253, filed on Oct. 20, 2020.

(51) Int. Cl. A61M 35/00; A61K 31/40; A61K 31/225

(52) U.S. Cl. CPC A61M 35/003; A61K 31/225; A61K 31/40

(57) ABSTRACT

Applicators for topical administration of anticholinergic compounds to a subject in need thereof include, in embodiments, a roll-on applicator, and a bottle that attaches to the roll-on applicator. Also disclosed herein are methods of treating conditions in a subject in need thereof by administering therapeutically effective anticholinergic compounds to the skin of the subject with an applicator disclosed herein.

### CROSS-REFERENCE TO RELATED APPLICATIONS

The present application claims priority for U.S. Provisional Application No. 63/094,253, filed Oct. 20, 2020, and entitled “ROLL-ON APPLICATORS AND METHODS FOR TREATING DERMAL CONDITIONS”. The present application incorporates the entire contents of that provisional application by reference.

### FIELD

The present disclosure sets forth roll-on applicators and methods for topical administration of a drug to a subject in need thereof. The present disclosure also sets forth methods of treating dermal conditions in a subject in need thereof by administering a drug to the skin of the subject with an applicator or method described herein.

### BACKGROUND

Topical medications have been used to treat or prevent many conditions. Most topical medications are applied directly to the skin, for instance by hand. For some medications, it is desirable to reduce unwanted side effects. For a few topical medications, disposable sheets, wipes, cloths, towelettes, or pads have been used for application. Some topical applicators have been made of absorbent material which carries the medication, so that wiping the skin treatment area with these applicators enables some transfer of medication.

Topical cloth applicators have been used successfully. For instance, the QBREXZA cloth has a generally favorable and established benefit to risk profile as a cloth presentation. QBREXZA cloth is generally well tolerated with infrequent adverse events that can be related to drug transfer to hands. As improvements, providing a no-touch presentation could decrease transfer-related adverse events and further improve the already favorable benefit-to-risk profile.

Conventional applicators, such as roll-on applicators, rub-on sticks, and aerosol spray cans, can also present difficulties in maintaining stability of a drug or drug product. For example, with liquid formulations, leakage, evaporation, and stability are concerns. Applicators should provide a meaningful shelf life (e.g. lasting multiple years), including little or no leakage or air exposure in storage, to reduce evaporation and/or degradation of formulations. In use, the applicators should also provide little or no leakage for the user. Leakage is a particular concern for formulations that are liquids, and specifically for those liquids that include alcohol. Further, the applicators should be appropriate for use on the skin with or without hair, for instance, on axillary skin, i.e. skin under the arms.

It would be desirable to provide an applicator and method that allow a user to dispense an amount of drug or drug product to the skin while reducing leakage, evaporation, and degradation.

### SUMMARY

These and other needs are addressed by the applicators and methods provided herein. In one aspect, embodiments are applicators for topical administration of a compound or pharmaceutical composition to a subject in need thereof. In certain embodiments, the applicators are roll-on applicators. In another aspect, methods administer the compound or pharmaceutical composition to skin of the subject with one of the described applicators. Useful compounds, compositions, methods of administration, and methods of treatment are described herein.

In aspects of the invention, an applicator for topical administration of a compound, or a pharmaceutically acceptable salt or composition thereof, to a subject in need thereof, comprises:
a. a bottle containing the compound, the bottle comprising bottle connector structure at an upper portion thereof and
b. a sleeve comprising an applicator member, at a top portion of the sleeve, for administering the compound, and sleeve connector structure to mate with the bottle connector structure to provide communication between the bottle and the applicator member to enable administration of the compound.

In aspects of the invention, the bottle connector structure and the sleeve connector structure comprise respective screw threads, snap fit structures, or press fit structures to the bottle and the sleeve. In some aspects, the mating provides a seal to reduce leakage and/or evaporation of the composition.

In aspects of the invention, the applicator member may comprise one or more rollers (including spheroids and ellipsoids that are rotatable about a major axis), made of one or more polymer materials selected from among polypropylene (including high-density polypropylene), polyethylene (including high-density polyethylene), polytetrafluoroethylene (PTFE), cyclic co-polymer, nylon, or polyethylene terephthalate (PET); stainless steel; or glass; disposed at the top portion of the sleeve. In aspects of the invention, the roller(s) may be sized to administer a desired amount of the compound. In some aspects, a gap between an inner diameter of the opening(s) sized to receive the roller(s), together with the viscosity of the compound being administered, facilitate the administration of the desired amount of the compound.

In aspects of the invention, the applicator also may comprise a sleeve cap which is screwed, snap fit, or press fit to the sleeve to provide a seal. In aspects of the invention, the bottle may have a bottle cap that is screwed, snap fit, or press fit onto the bottle to provide a seal, the bottle cap being removed prior to mating of the bottle with the sleeve.

In aspects of the invention, the bottle may have a reservoir that extends below the sleeve. In an embodiment, the reservoir may be opaque. In an embodiment, the reservoir may be translucent. In an embodiment, the reservoir may have a window comprising at least one translucent portion. The translucence of part or all of the reservoir may serve to alert the user to the amount of drug remaining to be administered.

In aspects of the invention, a neck of the bottle may be snap fit or press fit into the sleeve. In aspects of the invention, the bottle neck may be sized to fit snugly in the sleeve.

In aspects of the invention, a method for drug administration comprises the administration of a drug to the skin of a patient, wherein the drug is dispensed from the applicator of any one or a combination of aspects above.

In aspects of the invention, the applicator maintains stability of the composition comprising the anticholinergic compound. In aspects of the invention, the applicator provides weight loss of the composition in a range of less than 5%, less than 1%, for instance about 0.01 to 0.03%, by weight after two weeks at a temperature of about 25° C. and a relative humidity of about 60% with the applicator in a shelf-life configuration. In aspects of the invention, the applicator provides weight loss of the composition in a range of less than 5%, less than 1%, for instance about 0.04 to 0.06%, by weight after one month at a temperature of about 25° C. and a relative humidity of about 60% with the applicator in a shelf-life configuration. In aspects of the invention, the applicator provides weight loss of the composition in a range of less than 5%, less than 1%, for instance about 0.1 to 0.25% by weight after two weeks at a temperature of about 40° C. and a relative humidity of about 75% with the applicator in a shelf-life configuration. In aspects of the invention, the applicator provides weight loss of the composition in a range of less than 5%, less than 1%, for instance about 0.25 to 0.6%, by weight after one month at a temperature of about 40° C. and a relative humidity of about 75% with the applicator in a shelf-life configuration. In aspects of the invention, the applicator provides weight loss of the composition in a range of less than 5%, less than 1%, for instance about 0.0 to 0.3%, by weight after one week at ambient temperature. In aspects of the invention, the applicator provides weight loss of the composition in a range of less than 5%, less than 1%, for instance about 0.0 to 0.35%, by weight after two weeks at ambient temperature. In aspects of the invention, the applicator provides weight loss of the composition in a range of less than 5%, less than 1%, for instance about 0.0 to 0.05%, by weight after two weeks at a temperature of about 25° C. and a relative humidity of about 60% with the applicator in an in-use configuration. In aspects of the invention, the applicator provides weight loss of the composition in a range of less than 5%, less than 1%, for instance about 0.05 to 0.15%, by weight after two weeks at a temperature of 40° C. and a relative humidity of about 75% with the applicator in an in-use configuration. In aspects of the invention, the applicator provides weight loss of the composition in a range of less than 30%, for instance about 15 to 25%, by weight after two weeks at a temperature of 60° C. with the applicator in an in-use configuration.

In any of the aspects above, the drug is provided in the form of a solution, suspension, gel, thin gel, lotion, thin lotion, oil, low viscosity semi solid, or other form suitable for topical administration. In any of the aspects above, the drug is in a liquid solution or suspension. In any of the aspects above, the drug is a prescription medicine, an over-the-counter product, or any other substance for topical administration having at least one active ingredient.

In any of the aspects above, the drug is: for the treatment of wrinkles, brown spots or surface roughness; an anesthetic; for the treatment of acne; for the treatment of psoriasis; for the treatment of skin ulcers; for the treatment of diabetic foot ulcers; for the treatment or prevention of baldness; for the treatment of infection; for the treatment of warts; for the treatment of dermatosis; for the treatment of tinea pedis, tinea versicolor, tinea cruris, tine corporis, jock itch or ringworm; for the treatment of dermatitis; for the treatment of rosacea; for the treatment of lice; for the treatment of actinic keratosis; for the treatment of varicose veins; for the treatment of cancer; for the treatment of onychomycosis; for treatment of hyperhidrosis; for the prevention of sunburn or UV protection; a deodorant or an antiperspirant.

In any of the aspects above, the drug is: sunscreen, hydrocortisone, steroid, testosterone, estrogen, tretinoin; benzocaine, butamben, dibucaine, lidocaine, oxybuprocaine, pramoxine, proparacaine, proxymetacaine, or tetracaine; erythromycin, benzoyl peroxide, clindamycin, penederm, sodium sulfacetamide, adapalene or Tazorac; alefacept or Tazorac; becaplermin; minoxidil; tigecycline, clindamycin or butenafine; podofilox; betamethasone; luliconazole, terbinafine or terbinafine hydrochloride; tacrolimus; azelaic acid; ivermectin; ingenol mebutate; polidocanol; mechlorethamine; efinaconazole; glycopyrronium bromide; glycopyrronium tosylate; glycopyrronium tosylate monohydrate; sofpironium bromide; or an aluminum salt.

In certain embodiments, the applicators and methods are useful for delivering an effective amount of an anticholinergic compound to the skin of a subject. In certain embodiments, the applicators and methods are useful for treating hyperhidrosis. In certain embodiments, the applicators and methods are useful for treating axillary hyperhidrosis.

### BRIEF DESCRIPTION OF THE DRAWINGS

Embodiments in accordance with aspects of the invention now will be described in detail with reference to the accompanying drawings, in which:

FIG. 1 is a side view drawing of a bottle according to an embodiment;

FIGS. 2A to 2E are top view drawings the bottle according to embodiments;

FIG. 3 is a bottom view drawing of the bottle according to an embodiment;

FIG. 4 is a top view drawing of the bottle according to an embodiment;

FIGS. 5A and 5B are a side view and a top view, respectively, of a bottle according to an embodiment with a cap in place; FIGS. 5C and 5D are the views of FIGS. 5A and 5B, respectively, without the cap; FIG. 5E is a bottom view of a bottle according to an embodiment; and FIGS. 5F-H are a side, top, and bottom view, respectively, of a cap for the bottle according to an embodiment;

FIG. 6 is a side view of an applicator attachment or sleeve according to an embodiment;

FIG. 7 is an enlarged side view of an upper portion of the overall sleeve according to an embodiment;

FIG. 8 is an enlarged top view of the sleeve of FIG. 7, showing a roller that is part of the sleeve;

FIG. 9A-9C are bottom views of the sleeve according to an embodiment, with FIGS. 9B and 9C being enlarged views of the interior portion that FIG. 9A shows;

FIG. 10 is a side view of an assembly of the bottle and the sleeve according to an embodiment;

FIGS. 11A-11C are a side, top, and bottom view, respectively, of a cap of the sleeve according to an embodiment;

FIGS. 12A-12C are cross-sections of portions of a cap covering a roller according to various embodiments.

### DETAILED DESCRIPTION

Set forth herein are applicators and methods for administration of a compound or composition to the skin of a patient in need thereof.

### A. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures known in the art that are described or referenced herein are generally well understood and commonly employed using conventional methodologies by those skilled in the art.

As used herein, the singular forms “a,” “an,” and “the” include the plural referents unless the context clearly indicates otherwise.

As used herein, the term “about” refers to the stated value±10%, ±5%, or ±1%. For example, a value of “about 10” can encompass a range of 9 to 11. For logarithmic scales, the term “about” refers to the stated value±0.3 log units, or ±0.2 log units, or ±0.1 log units. For example, a value of “about pH 4.6” can encompass a pH range of 4.5-4.7.

As used herein, “treating” or “treatment” of hyperhidrosis refers, in certain embodiments, to ameliorating hyperhidrosis that exists in a subject. In some embodiments, “treating” or “treatment” includes ameliorating at least one physical parameter of hyperhidrosis, such as sweating. In some embodiments, “treating” or “treatment” includes modulating the hyperhidrosis. In some embodiments, “treating” or “treatment” includes delaying or preventing the onset of hyperhidrosis (e.g., a prophylactic treatment). In some embodiments, “treating” or “treatment” includes mitigating the incidence of episodes of hyperhidrosis, by periodic administration of a pharmaceutical composition according to the methods provided herein.

As used herein, the term “therapeutically effective amount” or “effective amount” refers to an amount of a composition provided herein that is useful for treating hyperhidrosis.

As used herein, the term “effective hyperhidrosis treatment” includes any treatment that may be used in treating hyperhidrosis. Any suitable effective hyperhidrosis treatment may be used in the methods provided herein. Illustrative suitable effective hyperhidrosis treatments include, for example, agents for the treatment of hyperhidrosis as described elsewhere in this disclosure (e.g., anticholinergic agents, metal salts, or toxins), microwave heating, iontophoresis, surgical removal of sweat glands, sympathectomy, ultrasound, and laser-based treatment. In particular embodiments, agent is a glycopyrronium compound.

As used herein, the terms “subject” and “patient” mean a mammalian subject. Exemplary subjects include, but are not limited to humans, monkeys, cows, horses, camels, goats and sheep. In certain embodiments, the subject is a human.

As used herein, the term “alkyl” refers to a monovalent and saturated hydrocarbon radical moiety. Alkyl is optionally substituted and can be linear, branched, or cyclic, i.e., cycloalkyl. Alkyl includes, but is not limited to, those having 1-20 carbon atoms, i.e., C₁₋₂₀ alkyl; 1-12 carbon atoms, i.e., C₁₋₁₂ alkyl; 1-8 carbon atoms, i.e., C₁₋₈ alkyl; 1-6 carbon atoms, i.e., C₁₋₆ alkyl; and 1-3 carbon atoms, i.e., C₁₋₃ alkyl. Examples of alkyl moieties include, but are not limited to methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl.

As used herein, the term “cycloalkyl” refers to a cyclic alkyl. Cycloalkyl is optionally substituted. Examples of cycloalkyl moieties include, but are not limited to, cyclopropyl, cyclobutyl, cyclopentyl, cyclohexyl, cycloheptyl, and cyclooctyl.

As used herein, “alkoxy” refers to a monovalent and saturated hydrocarbon radical moiety wherein the hydrocarbon includes a single bond to an oxygen atom and wherein the radical is localized on the oxygen atom, e.g. CH₃CH₂—O. for ethoxy. Alkoxy substituents bond to the compound which they substitute through this oxygen atom of the alkoxy substituent. Alkoxy is optionally substituted and can be linear, branched, or cyclic, i.e., cycloalkoxy. Alkoxy includes, but is not limited to, those having 1-20 carbon atoms, i.e., C₁₋₂₀ alkoxy; 1-12 carbon atoms, i.e., C₁₋₁₂ alkoxy; 1-8 carbon atoms, i.e., C₁₋₈ alkoxy; 1-6 carbon atoms, i.e., C₁₋₆ alkoxy; and 1-3 carbon atoms, i.e., C₁₋₃ alkoxy. Examples of alkoxy moieties include, but are not limited to methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, the term “alkoxycarbonyl,” refers to a monovalent and saturated hydrocarbon radical moiety wherein the hydrocarbon includes a single carbon bond to an oxygen atom, which is further bonded to a carbonyl, e.g., C(O). The oxygen atom is a bivalent atomic linker between the alkyl portion of the alkoxycarbonyl and the carbonyl. The radical in alkoxycarbonyl is localized on the carbon atom of the carbonyl which is bonded to an oxygen atom of an alkoxy e.g. CH₃CH₂—O—C.(O). Alkoxycarbonyl substituents bond to the compound which they substitute through this carbonyl carbon atom. Alkoxycarbonyl is optionally substituted and can be linear or branched. Alkoxycarbonyl includes, but is not limited to, those having 1-20 carbon atoms, i.e., C₁₋₂₀ alkoxycarbonyl; 1-12 carbon atoms, i.e., C₁₋₁₂ alkoxycarbonyl; 1-8 carbon atoms, i.e., C₁₋₈ alkoxycarbonyl; 1-6 carbon atoms, i.e., C₁₋₆ alkoxycarbonyl; and 1-3 carbon atoms, i.e., C₁₋₃ alkoxycarbonyl. Examples of alkoxy moieties include, but are not limited to methoxycarbonyl, and ethoxycarbonyl.

As used herein, the phrase “stereomerically pure,” refers to a particular stereoisomer of a compound which is present to a greater extent than other stereoisomers of that compound, e.g., the compound is present in diastereomeric excess or the compound is present in enantiomeric excess. In some embodiments, the stereomerically pure compounds described herein include 80% or greater, 85% or greater, 90% or greater, 95% or greater, or 97% or greater by weight of one stereoisomer of the compound. In some embodiments, the stereomerically pure compounds described herein include 80% or greater, 85% or greater, 90% or greater, 95% or greater, or 97% or greater by mole of one stereoisomer of the compound.

As used herein, unless specified otherwise, the percentage of an active ingredient refers to the weight percent of the active ingredient, excluding the weight of any counter-ions or water molecules in any salts and/or hydrates, in the composition. The person of skill should be able to convert readily these percentages when measuring salts and/or hydrates.

In the above definitions, “optionally substituted” indicates optional substitution with halogen, hydroxyl, methoxy, amino, or cycloalkyl. Unless specified otherwise, substituents are not further substituted.

As used herein, the phrase “pharmaceutically acceptable counter ion” refers to ions which retain the biological effectiveness and properties of the glycopyrronium base, which are not biologically or otherwise undesirable, and which carry an anionic charge. The glycopyrronium base forms salts by virtue of the presence of the quaternary ammonium thereon. The pharmaceutically acceptable counter ion may be prepared from inorganic or organic acids. Salts derived from inorganic acids include, but are not limited to, hydrochloric acid, hydrobromic acid, hydrogen fluoride, hydrogen iodide, sulfuric acid, nitric acid, phosphoric acid, and the like. Salts derived from organic acids include, but are not limited to, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. In some embodiments, the salt is derived from p-toluene sulfonic acid or hydrobromic acid.

### B. Applicators and Use

The following describes applicators and methods for the topical administration of a drug by a user. The drug can be any drug that a user (a patient or a caregiver) wishes to administer topically. In particular embodiments, the drug is provided in the form of a solution, suspension, gel, thin gel, lotion, thin lotion, oil, low viscosity semi solid, or other form suitable for topical administration. In certain embodiments, the drug is in a liquid solution or suspension. In certain embodiments, the drug is in a gel, for instance a thin gel. In certain embodiments, the drug is in a lotion, for instance a thin lotion. Throughout this specification, “thin” in reference to a solution, suspension, gel, lotion, semi-solid, or other form suitable for topical administration may be understood to mean “low viscosity”. In certain embodiments, the drug is in an oil. In certain embodiments, the drug is in the form of a low viscosity semi solid suspension or composition. The drug can be a prescription medicine, an over-the-counter product, or any other substance for topical administration.

In certain embodiments, the applicators are roll-on applicators. In certain embodiments, the applicators provide shelf life stability for a compound or composition contained within. In certain embodiments, the applicators provide little or no leakage of a compound or composition contained within, or of the medium in which the compound or composition is dispersed, even when the applicator is inverted. In certain embodiments, the applicators provide little or no air exposure to a compound or composition contained within. In certain embodiments, the applicators yield little or no evaporation of a compound or composition contained within, or of the medium in which the compound or composition is dispersed.

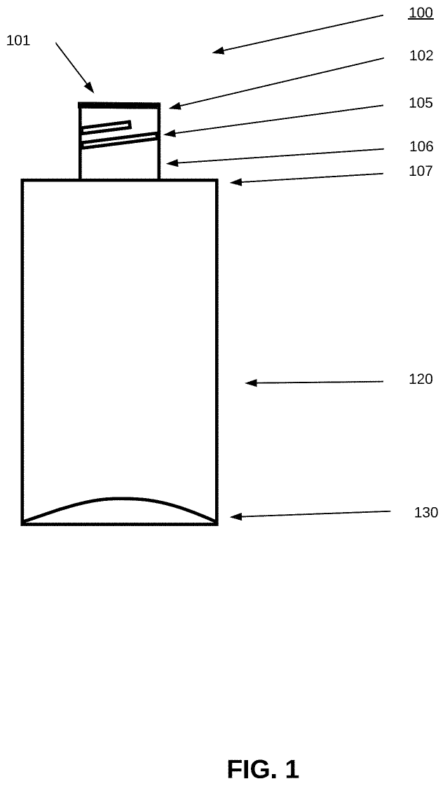

Referring now in more detail to the drawings for purposes of illustrating exemplary embodiments of the invention, wherein like reference numerals designate corresponding or like elements among the several views, FIG. 1 shows a side view of a drawing of bottle 100 that contains the formulation to be administered. In FIG. 1, threaded portion 105 is near a top of bottle 100, below opening 102. In an embodiment, an induction seal 101 fits over opening 102 to provide a seal to bottle 100 prior to dispensing of contents of the bottle. The threaded portion 105 is on a neck 106 of bottle 100. Threaded portion 105 enables bottle 100 to be attached to a sleeve, to be described below. Other attachment structures, including press fit and snap fit structures, will be known to ordinarily skilled artisans. With such structures, there may be one or more protruding portions on the outside of neck 106 in lieu of the threaded portion 105, to facilitate the snap fit or press fit. Such a protruding portion may extend around the circumference of neck 106. In embodiments, the one or more protruding portions (or a single circumferential portion) may extend around the inside of neck 106 rather than the outside.

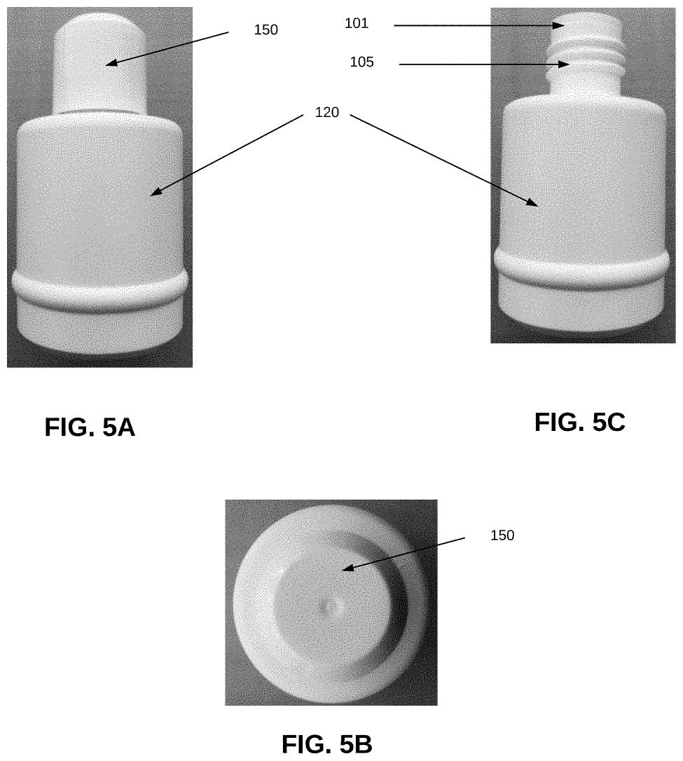

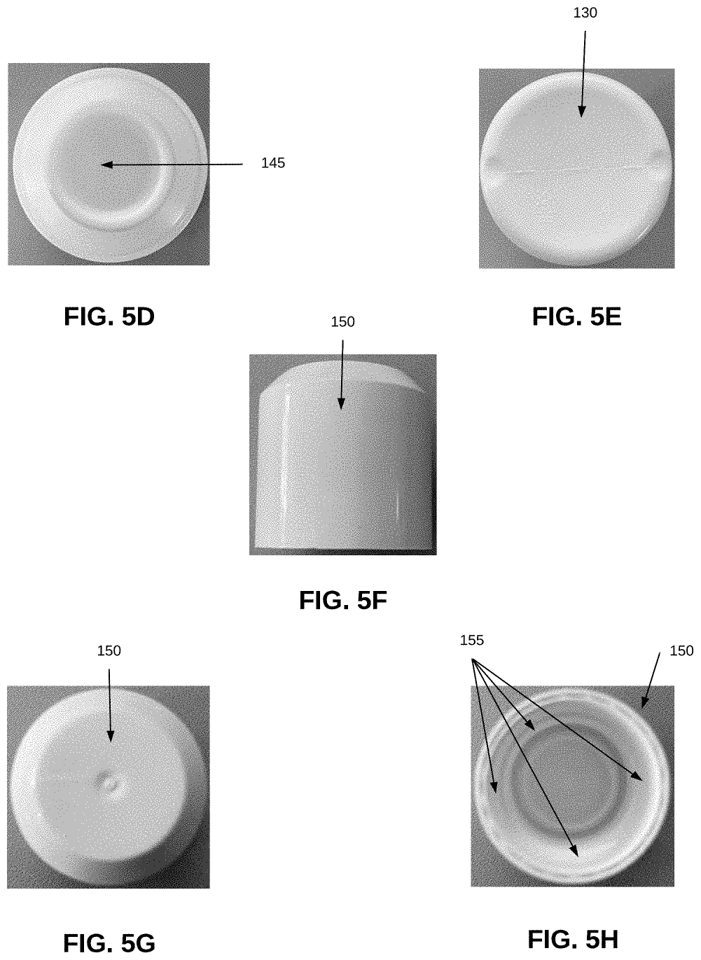

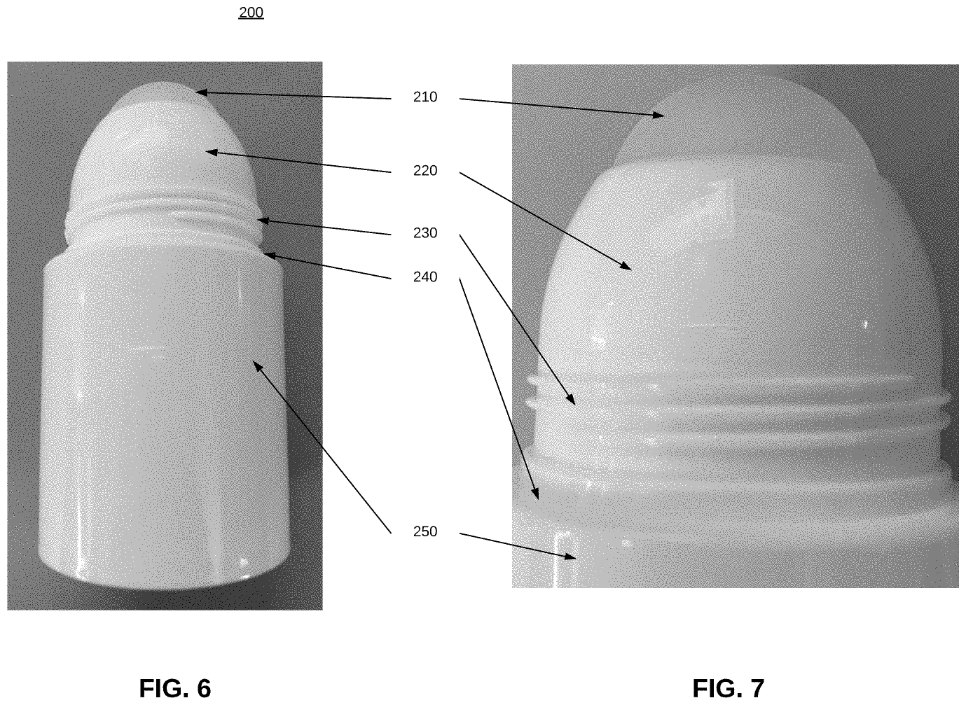

A shoulder portion 107 appears below the neck 106. When a bottle cap 150 (for example, FIGS. 5F-5H), is seated on bottle 100, a bottom of cap fits over shoulder portion 107 and seats on a bottom of the shoulder portion 107 on bottle 100. In an embodiment, reservoir 120 contains a majority of the volume of bottle 100. In an embodiment, as shown for example in FIGS. 5A and 5C (but not in FIG. 1), there may be a portion that extends around a circumference of reservoir 120. When a sleeve 200 (FIG. 6) is attached to bottle 100 in a manner to be described, a bottom portion of the sleeve 200 may seat on this portion. Alternatively, the portion may help the bottle 100 fit more snugly within sleeve 200 and avoid torque or other stress on neck 106. In FIG. 1, a concave portion 130 is at an underside of bottle 100, leaving a concave indentation at a bottom portion of reservoir 120.

The bottle shape in FIG. 1 is merely exemplary. In embodiments, the bottle 100 is sized to provide 30 to 60 dosages. The bottle shape may vary depending on configurations of sleeve 200, which in varying embodiments will enable bottle 100 to be screwed, snap fit, or press fit to attach to sleeve 200.

In an embodiment, bottle 100 may be constructed of polypropylene homopolymer (including high-density polypropylene) or polypropylene randomized copolymer, polyethylene (including high-density polyethylene), polytetrafluoroethylene (PTFE), cyclic co-polymer, nylon, or polyethylene terephthalate (PET). In an embodiment, reservoir 120 may be opaque. In an embodiment, reservoir 120 may be translucent. In an embodiment, a portion of reservoir 120 may be translucent, forming a window in reservoir 120. The translucence of part or all of reservoir 120 may facilitate the user's ability to observe an amount of drug remaining for administration.

In an embodiment in accordance with aspects of the invention, the bottle 100 may have a fill capacity of 5 to 70 mL, preferably 10 to 60 mL, more preferably 10 to 50 mL, still more preferably 10 to 40 mL, yet more preferably 10 to 30 mL, yet still more preferably 20 to 30 mL, and still more preferably either 20 to 25 mL or 25 to 30 mL, and in an embodiment, 25 mL. Overall height of bottle 100 may be 50 to 80 mm, preferably 60 to 80 mm, more preferably 70 to 80 mm, and still more preferably 75 to 80 mm. According to embodiments, a height of a cap portion at the top of bottle 100, in a vicinity of threaded portion 105, may be 15 to 20 mm, preferably 18 to 20 mm, more preferably 19 to 20 mm, and in an embodiment, 19.5 mm. According to various embodiments, a diameter of reservoir 120 may be 40 to 50 mm, preferably 45 to 50 mm, more preferably 45 to 47 mm. According to embodiments, a height of reservoir 120 may be 10 to 25 mm, preferably 15 to 20 mm, more preferably 15 to 17 mm, and in an embodiment, 16 mm.

Looking at dimensions at the top of bottle 100, according to embodiments an inner diameter of opening 102 at the top of bottle 100 may have an inner diameter of 10 to 15 mm, preferably 12 to 15 mm, more preferably 13 to 15 mm. According to embodiments, a diameter at an outer portion of a lip at the top of opening 102 may be 15 to 20 mm, preferably, 16 to 19 mm, more preferably 17 to 18 mm. According to embodiments, a diameter at the top of bottle 100 including threads 105 may be 15 to 20 mm, preferably 17 to 20 mm, more preferably 18 to 20 mm.

While exemplary dimensions have been provided in some instances, necessary acceptable manufacturing tolerances for all of the provided dimensions and measurements will be known to ordinarily skilled artisans.

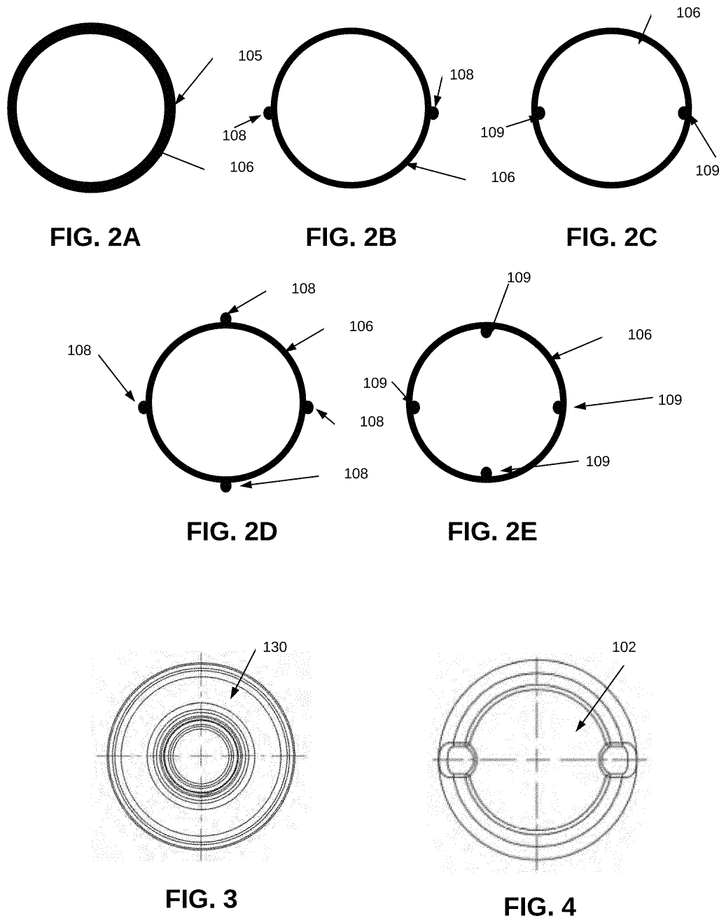

FIG. 2A shows an enlarged view of threaded portion 105 at a top of bottle 100, outside neck 106. FIG. 2B shows a pair of diametrically-opposed protrusions 108 outside of neck 106, and FIG. 2C shows a pair of diametrically-opposed protrusions 109 inside of neck 106. FIG. 2D shows two pairs of diametrically-opposed protrusions 108 outside of neck 106, and FIG. 2E shows two pairs of diametrically-opposed protrusions 109 inside of neck 106. Ordinarily skilled artisans will appreciate that other protrusion configurations and/or arrangements, facilitating a snap fit or a press fit, will be possible.

FIG. 3 shows a bottom view of bottle 100, including concave portion 130. FIG. 4 shows a top view of bottle 100, including opening 102.

FIGS. 5A and 5B show a side view and a top view, respectively, of a bottle 100 according to an embodiment. In FIG. 5A, the bottle 100 has a cap 150 covering a threaded portion 105 at the top of the bottle. In an embodiment, cap 150 may be constructed of polypropylene (PP) homopolymer (including high-density polypropylene) or polypropylene randomized copolymer, polyethylene (including high-density polyethylene), polytetrafluoroethylene (PTFE), cyclic co-polymer, nylon, or polyethylene terephthalate (PET).

FIG. 5B shows a top view of the bottle, including the cap 150 at the top covering the threaded portion 105. FIGS. 5C and 5D show a side view and a top view, respectively, of the bottle 100, without the cap 150, and showing the threaded portion 105 at the top of the bottle. FIG. 5C shows a location of induction seal 101. In an embodiment, a pitch of threaded portion 105 may be 3 mm. FIG. 5E shows a view of the bottom 125 of the bottle 100, also showing the concave portion 130. In any of the preceding embodiments, induction seal 101 may be made of a material such as polyvinylidene chloride (PVDC).

FIGS. 5F, 5G, and 5H are a side view, a top view, and a bottom view, respectively, of the cap 150 of FIGS. 5A and 5B. FIG. 5H shows an interior threaded portion 155. When the cap 150 is screwed onto bottle 100, the respective threaded portions 155 and 105 mate. In an embodiment, a pitch of threads in threaded portion 155 may be 3 mm, to match the pitch of threads in threaded portion 105. In an embodiment, when the bottle cap 150 is attached to the bottle 100, the respective threaded portions 155 and 105 engage each other sufficiently tightly to constitute a child resistant structure.

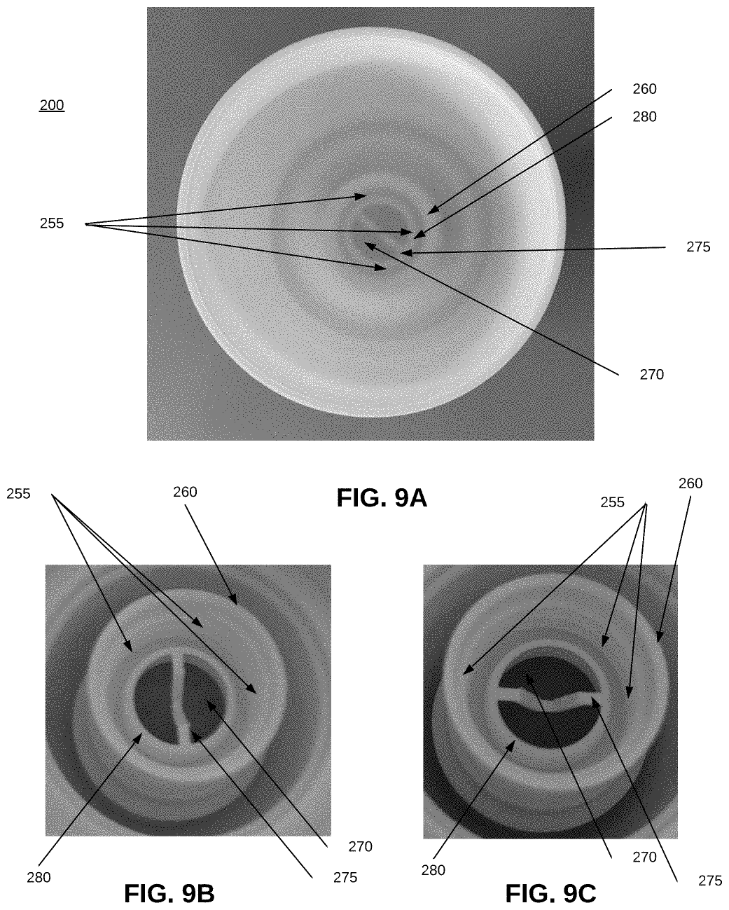

FIG. 6 shows a side view of a sleeve 200 into which bottle 100 fits. In embodiments, sleeve 200 may be constructed of polypropylene (PP) homopolymer (including high-density polypropylene) or polypropylene randomized copolymer, polyethylene (including high-density polyethylene), polytetrafluoroethylene (PTFE), cyclic co-polymer, nylon, or polyethylene terephthalate (PET). When fitting bottle 100 into sleeve 200, the cap 150 is removed so that the screw threads 105 on the bottle are exposed. These screw threads 105 mate with screw threads 255 (FIGS. 9A-9C) inside sleeve 200. In an embodiment, a pitch of threads in threaded portion 255 may be 3 mm, to match the pitch of threads in threaded portion 105. The thread pitch should be suitable to effect a good seal between mating threads to keep the medication within bottle 100 from evaporating, leaking, or drying out. In other embodiments, cap 150 may connect with bottle 100 via a snap fit or press fit, with complementary structures to the ones shown in FIGS. 2B-2E, to provide an a seal to reduce leakage and/or evaporation.

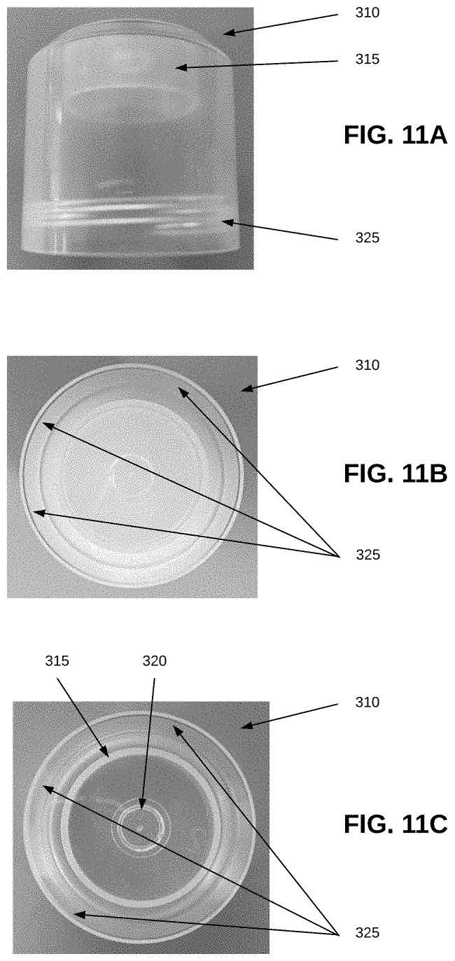

Looking more closely at FIG. 6, roller 210 is fit in place in tapered or curved portion 220 at the top. Screw threads 230 mate with screw threads 325 in cap 310 (FIG. 11A). When cap 310 is attached to sleeve 200, a bottom portion of cap 310 will seat on shoulder portion 240, between body 250 and screw threads 230 in sleeve 200. In an embodiment, cap 310 may be constructed of polypropylene (PP) homopolymer (including high-density polypropylene) or polypropylene randomized copolymer, polyethylene (including high-density polyethylene), polytetrafluoroethylene (PTFE), cyclic co-polymer, nylon, or polyethylene terephthalate (PET).

FIG. 7, which is an enlarged view of an upper portion of sleeve 200, shows all of the same components as FIG. 6, including roller 210, tapered or curved portion 220, screw threads 230, shoulder portion 240, and an upper portion of body 250.

In FIGS. 6 and 7, body 250 is smooth and has a uniform diameter that may taper slightly. In an embodiment, part or all of body 250 may be rough or textured for easier gripping. In an embodiment, body 250 also may have a narrower portion near the middle, also to facilitate gripping. In an embodiment, body 250 may have finger grips embedded therein, again to facilitate gripping. In an embodiment, part or all of body 250 may be rough or textured, and body 250 may have a portion with a narrower diameter near the middle, and/or finger grips, to make gripping even easier.

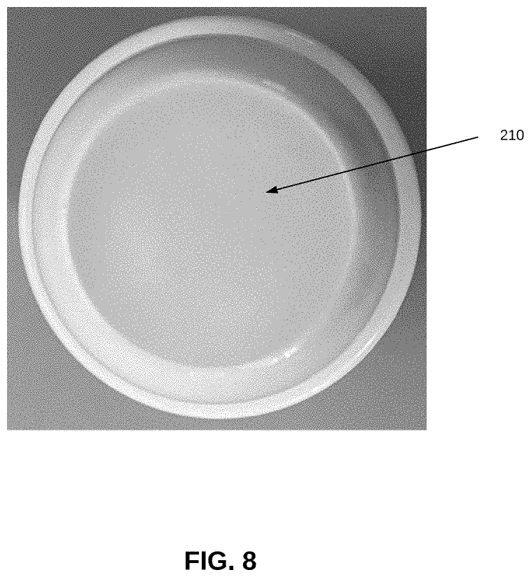

FIG. 8 shows a top view of sleeve 200 and an enlarged view of roller 210. In an embodiment, roller 210 is made of polypropylene (PP) homopolymer (including high-density polypropylene), polyethylene (including high-density polyethylene), polytetrafluoroethylene (PTFE), cyclic co-polymer, nylon, or polyethylene terephthalate (PET), stainless steel, or glass. In an embodiment, the roller 210 may be 10 to 30 mm in diameter, preferably 10 to 25 mm, more preferably 15 to 25 mm, still more preferably 20 to 25 mm, yet more preferably 23 to 25 mm. The size may depend on the medication being applied, the amount of medication being applied, and the area to which the medication is being applied. In an embodiment, the roller 210 is wider than an opening at the top of bottle 100, providing a larger surface area so that a user only has to roll the applicator one or a few times in order to cover the area to which medication is to be applied. In an embodiment, tolerances among roller 210, sleeve 200, and bottle 100 are tight, to keep air out and reduce leakage and/or evaporation. Where the medication to be applied has higher amounts of volatile ingredients, such as ethanol, it would be particularly desirable to avoid evaporation.

According to other embodiments, the roller 210 may be made of other suitable materials, such as stainless steel or glass. In other embodiments, instead of a single roller 210, there may be multiple such rollers at the top of sleeve 200. These multiple rollers may be made of PP (including high-density polypropylene), polyethylene (including high-density polyethylene), polytetrafluoroethylene (PTFE), cyclic co-polymer, nylon, or polyethylene terephthalate (PET), or stainless steel, or glass, or other suitable materials. The multiple rollers may be arranged as desired at the top of sleeve 200.

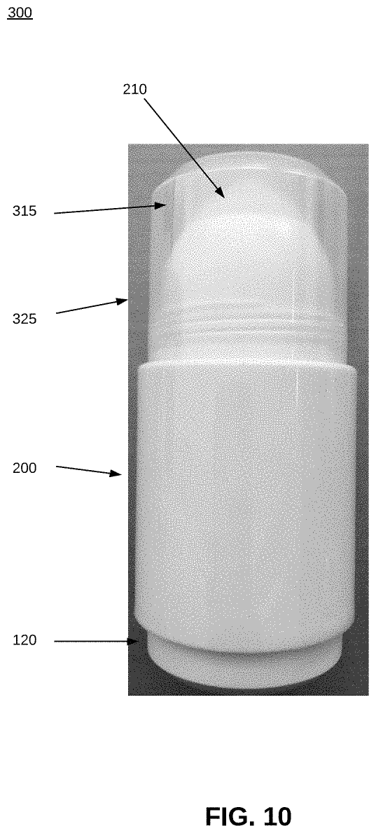

FIGS. 9A-9C show an interior of sleeve 200, and views of screw threads 255 that mate with screw threads 105 at the top of bottle 100 when the sleeve 200 and bottle 100 are assembled into a roll-on applicator 300 (FIG. 10). FIGS. 9B and 9C are enlarged views of the sleeve interior 200 of FIG. 9A, and show more clearly an optional piece 275 that connects to opposing sides of opening 270. FIGS. 9A-9C show a neck 260 extending downwardly from an upper portion of sleeve 200, below roller 210, FIGS. 9A-9C also show a lip 280 that extends from an upper portion of an interior of sleeve 200. In an embodiment, when an upper portion of bottle 200—in an embodiment, threads 105 (FIG. 1, 2A)—is mated with the screw threads 255 in an interior of sleeve 200, the portion of the top of bottle 100 with threads 105 fit between the lip 280 and the neck 260. As a result, when the threads 255 and 105 are mated, lip 280 extends into opening 145 of bottle 200, improving the connection between bottle 100 and sleeve 200.

In an embodiment, bottle 100 and sleeve 200 attach via mating screw threads, as just discussed. In other embodiments, the bottle 100 and sleeve 200 may be snap fit or press fit.

FIG. 10 shows a roll-on applicator assembly 300 comprising bottle 100 and sleeve 200, with bottle screwed into sleeve 200. When bottle 100 is screwed into sleeve 200 to form assembly 300, the opening 145 (FIG. 5D) at the top of bottle 100 and the opening 270 (FIGS. 9A-9C) near the top of sleeve 200 enable fluid communication between an interior of bottle 100 and roller 210 at the top of sleeve 200 in the assembly 300, enabling medication to be applied via the roller 210. In an embodiment, as FIG. 10 shows, a portion of reservoir 120 of bottle 100 may be visible beneath sleeve 200.

According to different embodiments, the roll-on applicator 300 can provide dosages of 0.1 to 1.0 g, 0.1 to 0.9 g, or 0.1 to 0.8 g. In different embodiments, the dosage range per use may be 0.1 to 0.7 g, 0.1 to 0.6 g, 0.1 to 0.5 g, 0.1 to 0.4 g, 0.1 to 0.3 g, 0.2 to 0.8 g, 0.3 to 0.8 g, 0.4 to 0.8 g, 0.1 g, 0.2 g, 0.25 g, 0.3 g, 0.4 g, 0.5 g, 0.6 g, 0.7 g or 0.8 g. For a given use, the just-mentioned dosage ranges and amounts per use can be delivered in multiple operations of the applicator 300, or in a single operation of the applicator 300 at the area to which medication is to be applied. The required number of operations of the applicator 300 to administer a given dose can be a function of the degree of communication between the interior 135 of reservoir 120 and roller(s) 210. That degree of communication can depend on the size of openings 145 and 270, as well as on the viscosity of the medication in the reservoir 120. In certain embodiments, the number of operations of the applicator 300 is one, two, three, four, five, six, seven, eight, nine, or ten, or more.

FIGS. 11A-11C show a side, top, and bottom view of cap 310 on applicator 300/sleeve 200. FIG. 11A shows threads 325 which mate with threads 230 on sleeve 200. Cap 310 provides a seal for applicator 300 after bottle 100 containing medication is mated with sleeve 200. In an embodiment, cap 310 may engage or surround or cover roller(s) 210 via covering portion 315 to prevent leakage or drying out of medication in applicator 300. FIG. 11C also shows covering portion 315 in engagement with roller 210.

In an embodiment, cap 310 may be constructed of polypropylene (PP) homopolymer (including high-density polypropylene) or polypropylene randomized copolymer, polyethylene (including high-density polyethylene), polytetrafluoroethylene (PTFE), cyclic co-polymer, nylon, or polyethylene terephthalate (PET).

In an embodiment, part or all of cap 310 may be rough or textured for easier gripping. In an embodiment, cap 310 also may have a narrower portion near the middle, also to facilitate gripping. In an embodiment, cap 310 may have finger grips embedded therein, again to facilitate gripping. In an embodiment, part or all of cap 310 may be rough or textured, and cap 310 may have a portion with a narrower diameter near the middle, and/or finger grips, to make gripping even easier.

In an embodiment, a round circular feature 320 (FIG. 11C) extends downwardly from a top interior portion of cap 310. The circular feature 320 (also referred to as a cap ring) can compress the roller 210 against tapered or curved portion 220 to provide a leak-proof seal between the roller 210 and the sleeve 220. In embodiments, a geometry devised among feature 320, the roller 210, and curved portion 220 can be adjusted in an optimal fashion to seal a gap between the roller 210 and an interior of sleeve 200.

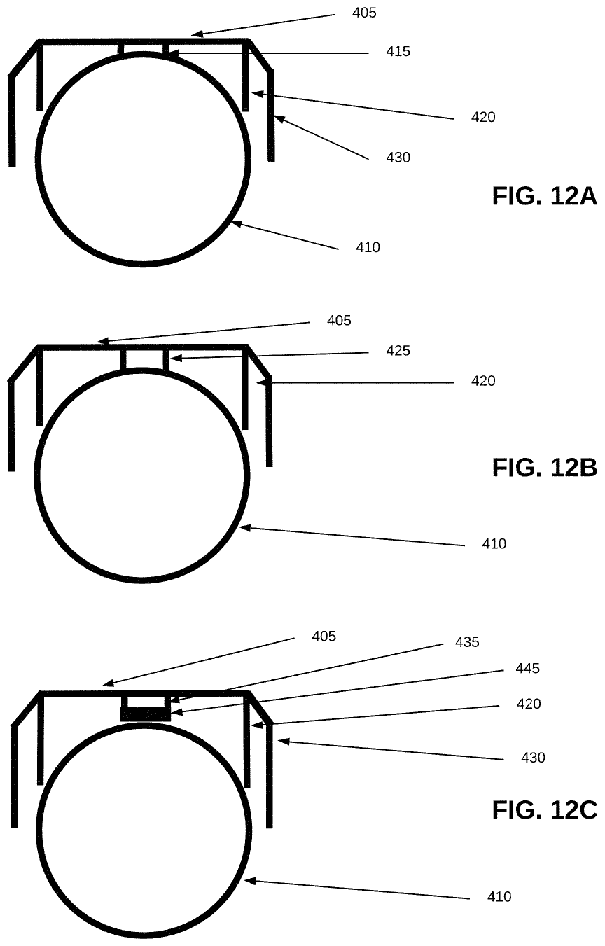

FIGS. 12A-12C show cutaway views of different cap configurations. FIG. 12A shows a cap configuration with a cap ring 415 extending downwardly from a cap top portion 405. In an embodiment, cap ring 415 extends 0.5 mm from the cop top portion 405. In some circumstances, cap ring 415 may not extend far enough down from cap top portion 405 to press roller 410 down against ring structure (not shown) to prevent leakage. In an embodiment, an outer ring 420 may extend between cap ring 415 and an outer portion 430 of the cap.

FIG. 12B shows structure similar to FIG. 12A, except that cap ring 425 extends farther down from cap top 405, for example, approximately 1 mm, and can exert more pressure against the roller 410 toward ring structure (not shown) to prevent leakage more effectively. FIG. 12C shows structure similar to FIGS. 12A and 12B, except that cap ring 435, which may extend the same amount as cap ring 415 or cap ring 425, or in some embodiments a different amount, has a compressible portion 445 attached to it. Compressible portion 445 should not be absorbent, to avoid it absorbing compound, but compressible portion 445 should be soft to enable additional pressure against roller 410. Compressible portion 445 may be in the shape of a cylinder, a disk, or a ring. In an embodiment, compressible portion 445 may have a sort of T-shape, with the stem of the T fitting into the cap ring 415 or 425.

In an embodiment, the cap may be screwed onto the sleeve via screw threads on the cap and screw threads on the sleeve, so that a neck on the sleeve receives the cap. In an embodiment, the cap may be a child resistant cap, requiring that a user press down on the cap before turning it to remove it. As normally engaged, the cap will seat such that the threads are in tight engagement. When the cap is pressed down, the threads disengage, enabling the cap to be unscrewed. Any of the cap ring configurations in FIGS. 12A-12C, including compressible portion 445, may facilitate the tight engagement of the threads of the cap and the sleeve.

In use, the bottle 100 may be left in mating connection with sleeve 200 to keep the roll-on assembly 300 intact until the medication in reservoir 300 is consumed. In that event, the user would simply discard cap 150. Alternatively, depending on the medication to be administered, or on frequency of administration, or on some other criteria with which ordinarily skilled artisans would be familiar, it may be useful or worthwhile to disconnect bottle 100 from sleeve 200, reattach cap 150 to bottle 100, and store bottle 100 until the next administration. For example, during air travel, changes in air pressure can cause leakage. Disconnecting bottle 100 from sleeve 200 and reattaching cap 150 to bottle 100 in such circumstances can help to preserve the compound until once again in a more normal pressure environment.

### C. Methods of Administration and Treatment

The applicators can be used to store and/or deliver a compound or composition for any use deemed suitable to the practitioner of skill. Useful compounds and compositions are described herein. The administration can be for any purpose deemed suitable by the practitioner of skill. In certain embodiments, the methods are for therapy. In certain embodiments, the methods are for prophylaxis. In certain embodiments, the methods are for therapy and for prophylaxis. In particular embodiments, the applicators are for treating or preventing a disease or disorder in a subject in need thereof.

The subject can be any subject deemed suitable by the practitioner of skill. In certain embodiments, the subject is a mammal. In certain embodiments, the subject is a human. In certain embodiments, the subject is an adult human. In certain embodiments, the subject is a pediatric human.

In certain embodiments, the compound is a drug. In certain embodiments, the compound is in the form of a pharmaceutical composition. In certain embodiments, the drug is for the treatment of wrinkles, brown spots or surface roughness. In certain embodiments, the drug is tretinoin. In certain embodiments, the drug is an anesthetic. In certain embodiments, the drug is benzocaine, butamben, dibucaine, lidocaine, oxybuprocaine, pramoxine, proparacaine, proxymetacaine, or tetracaine. In certain embodiments, the drug is for the treatment of acne. In certain embodiments, the drug is erythromycin, benzoyl peroxide, clindamycin, penederm, tretinoin, sodium sulfacetamide, adapalene or tazorac. In certain embodiments, the drug is for the treatment of psoriasis. In certain embodiments, the drug is alefacept or tazorac. In certain embodiments, the drug is for the treatment of skin ulcers such as diabetic foot ulcers. In certain embodiments, the drug is becaplermin. In certain embodiments, the drug is for the treatment or prevention of baldness. In certain embodiments, the drug is minoxidil. In certain embodiments, the drug is for the treatment of infection. In certain embodiments, the drug is tigecycline, clindamycin or butenafine. In certain embodiments, the drug is for the treatment of warts. In certain embodiments, the drug is podofilox. In certain embodiments, the drug is for the treatment of dermatosis. In certain embodiments, the drug is betamethasone. In certain embodiments, the drug is for the treatment of tinea pedis, tinea versicolor, tinea cruris, tine corporis, jock itch or ringworm. In certain embodiments, the drug is luliconazole, terbinafine or terbinafine hydrochloride. In certain embodiments, the drug is for the treatment of dermatitis. In certain embodiments, the drug is tacrolimus. In certain embodiments, the drug is for the treatment of rosacea. In certain embodiments, the drug is azelaic acid. In certain embodiments, the drug is for the treatment of lice. In certain embodiments, the drug is ivermectin. In certain embodiments, the drug is for the treatment of actinic keratosis. In certain embodiments, the drug is ingenol mebutate. In certain embodiments, the drug is for the treatment of varicose veins. In certain embodiments, the drug is polidocanol. In certain embodiments, the drug is for the treatment of cancer. In certain embodiments, the drug is mechlorethamine. In certain embodiments, the drug is for the treatment of onychomycosis. In certain embodiments, the drug is efinaconazole. In certain embodiments, the drug is a steroid. In certain embodiments, the drug is testosterone. In certain embodiments, the drug is estrogen. In certain embodiments, the drug is nicotine. In certain embodiments, the drug is scopolamine.

In certain embodiments, the drug is for treatment of hyperhidrosis. In certain embodiments, the drug is glycopyrrolate or glycopyrronium bromide. In certain embodiments, the drug is glycopyrronium tosylate. In certain embodiments, the drug is glycopyrronium tosylate monohydrate. In certain embodiments, the drug is sofpironium bromide. In certain embodiments, the drug is an antiperspirant, for instance an aluminum salt.

The applicators and methods can be for administration to any region of the skin. In particular embodiments, the administration is to one or more axilla. In particular embodiments, the administration is to one or more hands. In particular embodiments, the administration is to one or more palms. In particular embodiments, the administration is to one or more feet. In particular embodiments, the administration is to one or more foot soles. In particular embodiments, the administration is to the face. In particular embodiments, the administration is to the forehead. In particular embodiments, the administration is to the back. In particular embodiments, the administration is to the lower back. In particular embodiments, the administration is to the upper back. In particular embodiments, the administration is to the genitals. In certain embodiments, the therapeutic compounds target site of activity is the epidermal or dermal layer of the skin, or in tissue with or adjacent to sweat glands.

In certain embodiments, the methods are for treating or preventing hyperhidrosis in a subject in need thereof. In certain embodiments, the methods are for treating palmar hyperhidrosis in a subject in need thereof. In certain embodiments, the methods are for treating plantar hyperhidrosis in a subject in need thereof. In certain embodiments, the methods are for preventing palmar hyperhidrosis in a subject in need thereof. In certain embodiments, the methods are for preventing plantar hyperhidrosis in a subject in need thereof.

In certain embodiments, the compound is an anticholinergic compound. The practitioners of skill will recognize that such methods are useful for treating any cholinergic disease or disorder where topical administration is useful. In certain embodiments, the anticholinergic compound is selected from the group consisting of a glycopyrronium compound, a sofpironium compound, propantheline, oxybutynin, methantheline, and benztropine. In particular embodiments, the compound is glycopyrronium bromide. In particular embodiments, the compound is glycopyrronium tosylate. In particular embodiments, the compound is glycopyrronium tosylate monohydrate. In particular embodiments, the compound is sofpironium bromide. In each embodiment, the compound can be administered itself, or as a pharmaceutically acceptable salt or solvate. In certain embodiments, the compound is administered in a pharmaceutical composition further comprising one or more pharmaceutically acceptable carriers, excipients, or diluents. Particular useful compounds are described in the sections below.

The administration can be in any dose deemed suitable by the practitioner of skill. In certain embodiments, the compound is an anticholinergic compound, and the dose per use is from 1 mg to 5000 mg. In certain embodiments, the compound is an anticholinergic compound, and the dose is from 1 mg to 4000 mg. In certain embodiments, the compound is an anticholinergic compound, and the dose is from 1 mg to 3000 mg. In certain embodiments, the compound is an anticholinergic compound, and the dose is from 1 mg to 2000 mg. In certain embodiments, the compound is an anticholinergic compound, and the dose is from 1 mg to 1000 mg. In certain embodiments, the compound is an anticholinergic compound, and the dose is from 1 mg to 500 mg. In certain embodiments, the compound is an anticholinergic compound, and the dose is from 1 mg to 250 mg. In certain embodiments, the compound is an anticholinergic compound, and the dose is from about 1 mg to about 150 mg. In certain embodiments, the compound is an anticholinergic compound, and the dose is from about 1 mg to about 125 mg. In certain embodiments, the compound is an anticholinergic compound, and the dose is from about 1 mg to about 100 mg. In certain embodiments, the compound is an anticholinergic compound, and the dose is from about 1 mg to about 75 mg. In certain embodiments, the compound is an anticholinergic compound, and the dose is from about 1 mg to about 50 mg. In certain embodiments, the compound is an anticholinergic compound, and the dose is from about 1 mg to about 25 mg. In certain embodiments, the compound is glycopyrronium tosylate monohydrate, and the dose is from about 1 mg to about 40 mg. In certain embodiments, the compound is sofpironium bromide, and the dose is from about 10 mg to about 125 mg. As used herein, the term anticholinergic compound includes pharmaceutically acceptable salts and solvates, for instance, glycopyrronium bromide, glycopyrronium tosylate, glycopyrronium tosylate monohydrate, or sofpironium bromide.

The administration can be for any length of time deemed suitable by the practitioner of skill. In certain embodiments, the applicator may be used continuously a number of times, for durations of 1-10 minutes, 1-5 minutes, or for about 1 minute, or for about 15 seconds or less, as appropriate to administer the desired dose.

In certain embodiments, the dose is delivered by as few as 1, 2, 3, 4, or 5, and as many as 25, 30, 35, 40, 45, 50, 55, or 60 rolls of the applicator. In certain embodiments, the dose is delivered by as few as 4, 5, or 6, and as many as 10, 11, 12, 13, 14, 15, or 20 rolls of the applicator. In certain embodiments, instructions provided with applicator direct or guide the user to the number and form of applications to yield the intended dosage. Advantageously, multiple applications can allow an error on the part of the user to transfer less extra drug. For instance, with 10 intended applications, one extra application would be expected to transfer only about 10% additional drug.

The administration can be repeated as deemed needed by the practitioner of skill. In certain embodiments, the administration is once per day. In certain embodiments, the administration is twice per day. In certain embodiments, the administration is three times per day. In certain embodiments, the administration is four times per day. In certain embodiments, the administration is 2-3 times per day for a few days followed by once per day for a few days. The number of days can be determined by the practitioner of skill. In certain embodiments, the administration is 2-3 times per day for 2-3 days followed by once per day for 4-5 days.

In certain embodiments, the administration comprises a single application of the applicator and no further administration steps are needed. For instance, for administration to the axillary skin, one or more applications of the applicator can be sufficient in certain embodiments. In certain embodiments, the administration with the applicator is followed by occlusion of the skin surface at the site of administration. In certain embodiments, occlusion of the skin surface enhances administration to palmar skin. In certain embodiments, occlusion of the skin surface enhances administration to plantar skin. The occlusion can be with any material and for any length of time deemed suitable by the practitioner of skill.

In certain embodiments, the material is fully occlusive. In certain embodiments, the material is partially occlusive or semi-occlusive. In certain embodiments, the material is cotton. In certain embodiments, the material is 95-100% cotton. In certain embodiments, the material is about 100% cotton. In certain embodiments, the material is nitrile. In certain embodiments, the material is polyethylene, for instance Saran Wrap. In certain embodiments, certain embodiments, the material is latex. In certain embodiments, the material is plastic. In certain embodiments, the material is butyl rubber. In certain embodiments, the material is neoprene. In certain embodiments, the material is polyester. In certain embodiments, the material is wool. In certain embodiments, the material is rayon. In certain embodiments, the material is acrylic fiber.

The occluding can be for any length of time deemed suitable by the practitioner of skill. In certain embodiments, the occluding is for 0.5-12 hours, 1-10 hours, 1-8 hours, or 1-5 hours. In certain embodiments, the occluding is for about 8 hours. In certain embodiments, the occluding is overnight.

The administration can continue for any length of time deemed suitable by the practitioner of skill. In certain embodiments, the administration is for one day. In certain embodiments, the administration is for one week. In certain embodiments, the administration is for two weeks. In certain embodiments, the administration is for three weeks. In certain embodiments, the administration is for four weeks. In certain embodiments, the administration is for up to and/or including eight weeks. In certain embodiments, the administration continues as needed while symptoms are evident. In certain embodiments, the administration continues as needed to prevent symptoms. In certain embodiments, the administration is long-term, for instance as needed by the patient or as directed by a practitioner of skill.

The administration can be at any interval deemed suitable by the practitioner of skill. In certain embodiments, the administration is every day. In certain embodiments, the administration is every other day. In certain embodiments, the administration is four days per week. In certain embodiments, the administration is three days per week. In certain embodiments, the administration is two days per week. In certain embodiments, the administration is one day per week.

The administration can be monitored by any technique deemed suitable by the practitioner of skill. For hyperhidrosis, the administration can be followed by one or more questionnaires known to the practitioner of skill. In certain embodiments, treatment of hyperhidrosis can be monitored by the Hyperhidrosis Disease Severity Scale (HDSS). See Solish et al., Dermatol. Surg., 2007, 33:908-923, incorporated by reference in its entirety. In certain embodiments, treatment of hyperhidrosis can be monitored by the Axillary Sweating Daily Diary (ASDD). See Nelson et al., 2019, J. Patient Rep Outcomes 3(1):59, incorporated by reference in its entirety. In certain embodiments, the Axillary Sweating Daily Diary (ASDD) can be adapted for palmar, plantar, or facial/cranial assessment. In embodiments, treatment can be facilitated by age appropriate individuals. In the case of children or people of sufficiently advanced age, for example, a caregiver may administer the treatment. Other individuals may administer their own treatment. The ASDD can be modified to cater to different age groups.

### D. Compounds and Pharmaceutical Compositions

In the methods described herein, the compound can be any compound deemed suitable for palmar or plantar administration by the practitioner of skill. In certain embodiments, the compound is an anticholinergic compound. In certain embodiments, the compound is effective for the treatment of hyperhidrosis.

In certain embodiments, the compound is a drug. In certain embodiments, the compound is in the form of a pharmaceutical composition. In certain embodiments, the drug is for the treatment of wrinkles, brown spots or surface roughness. In certain embodiments, the drug is tretinoin. In certain embodiments, the drug is an anesthetic. In certain embodiments, the drug is benzocaine, butamben, dibucaine, lidocaine, oxybuprocaine, pramoxine, proparacaine, proxymetacaine, or tetracaine. In certain embodiments, the drug is for the treatment of acne. In certain embodiments, the drug is erythromycin, benzoyl peroxide, clindamycin, penederm, tretinoin, sodium sulfacetamide, adapalene or tazorac. In certain embodiments, the drug is for the treatment of psoriasis. In certain embodiments, the drug is alefacept or tazorac. In certain embodiments, the drug is for the treatment of skin ulcers such as diabetic foot ulcers. In certain embodiments, the drug is becaplermin. In certain embodiments, the drug is for the treatment or prevention of baldness. In certain embodiments, the drug is minoxidil. In certain embodiments, the drug is for the treatment of infection. In certain embodiments, the drug is tigecycline, clindamycin, or butenafine. In certain embodiments, the drug is for the treatment of warts. In certain embodiments, the drug is podofilox. In certain embodiments, the drug is for the treatment of dermatosis. In certain embodiments, the drug is betamethasone. In certain embodiments, the drug is for the treatment of tinea pedis, tinea versicolor, tinea cruris, tine corporis, jock itch or ringworm. In certain embodiments, the drug is luliconazole, terbinafine or terbinafine hydrochloride. In certain embodiments, the drug is for the treatment of dermatitis. In certain embodiments, the drug is tacrolimus. In certain embodiments, the drug is for the treatment of rosacea. In certain embodiments, the drug is azelaic acid. In certain embodiments, the drug is for the treatment of lice. In certain embodiments, the drug is ivermectin. In certain embodiments, the drug is for the treatment of actinic keratosis. In certain embodiments, the drug is ingenol mebutate. In certain embodiments, the drug is for the treatment of varicose veins. In certain embodiments, the drug is polidocanol. In certain embodiments, the drug is for the treatment of cancer. In certain embodiments, the drug is mechlorethamine. In certain embodiments, the drug is for the treatment of onychomycosis. In certain embodiments, the drug is efinaconazole.

In certain embodiments, the drug is an anticholinergic agent. In certain embodiments, the anticholinergic agent is selected from a glycopyrronium compound, propantheline, oxybutynin, methantheline, benztropine, and sofpironium bromide (BBI-4000; Brickell Biotech, Inc.). In some embodiments, the agent is a glycopyrronium compound. In some embodiments, the glycopyrronium compound is glycopyrronium tosylate. In some embodiments, the glycopyrronium compound is glycopyrronium tosylate monohydrate. In some embodiments, the glycopyrronium compound is glycopyrronium bromide. In certain embodiments, the compound is sofpironium bromide.

In some embodiments, the glycopyrronium compound is threo-glycopyrronium tosylate monohydrate?. In some embodiments, the glycopyrronium compound is threo-glycopyrronium bromide.

In some embodiments, the glycopyrronium compound comprises threo-glycopyrronium and erythro-glycopyrronium, wherein the threo-glycopyrronium is at least 95% of the total glycopyrronium content of the composition and the erythro-glycopyrronium is less than 5% of the total glycopyrronium content of the composition.

In certain embodiments, the method comprises administration of a compound of Formula (I):

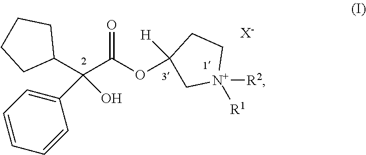

In Formula (I), R¹ and R² are each, independently in each instance, selected from alkyl and alkyl substituted with alkoxycarbonyl; and X⁻ is an anion. In certain embodiments, X⁻ is bromide. In certain embodiments, X⁻ is tosylate. In certain embodiments, alkyl is not further substituted, and alkoxycarbonyl is not substituted. In certain embodiments, the X⁻ is tosylate and the compound is a monohydrate.

In certain embodiments, the method comprises administration of a compound of Formula (I)

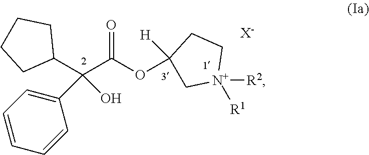

wherein R¹ and R² are each, independently in each instance, selected from alkyl and alkyl substituted with alkoxycarbonyl; the stereochemical configuration about the carbon atoms indicated by 2 and 3′ is a threo mixture, i.e. R/S and SIR; and X⁻ is an anion. In certain embodiments, X⁻ is bromide. In certain embodiments, X⁻ is tosylate. In certain embodiments, alkyl is not further substituted, and alkoxycarbonyl is not substituted. In certain embodiments, the X⁻ is tosylate and the compound is a monohydrate.

In certain embodiments, the method comprises administration of a compound of Formula (I)

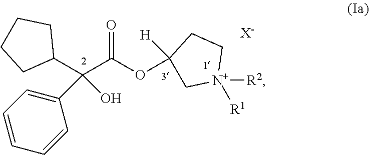

wherein R¹ and R² are each, independently in each instance, selected from alkyl and alkyl substituted with alkoxycarbonyl; the stereochemical configuration about the carbon atom indicated by 2 is R; the stereochemical configuration about carbon atom indicated by 3′ is R; and X⁻ is an anion. In certain embodiments, X⁻ is bromide. In certain embodiments, X⁻ is tosylate. In certain embodiments, alkyl is not further substituted, and alkoxycarbonyl is not substituted.

In certain embodiments, R¹ is alkyl. In certain embodiments, R¹ is methyl, ethyl, n-propyl, i-propyl, n-butyl, t-butyl, i-butyl, n-pentyl, or i-pentyl. In some examples, R¹ is methyl or ethyl. In certain embodiments, R¹ is methyl. In other examples, R¹ is ethyl.

In certain embodiments, R² is alkyl. In certain embodiments, R² is methyl, ethyl, n-propyl, i-propyl, n-butyl, t-butyl, i-butyl, n-pentyl, or i-pentyl. In certain embodiments, R² is methyl or ethyl. In certain embodiments, R² is methyl. In certain embodiments, R² is ethyl. In some examples, both R¹ and R² are methyl.

In certain embodiments, R¹ is alkyl substituted with alkoxycarbonyl. In certain embodiments, R¹ is methyl substituted with alkoxycarbonyl. In certain embodiments, R¹ is —CH₂C(O)OCH₂CH₃. In certain embodiments, R² is alkyl. In certain embodiments, R² is methyl or ethyl. In certain embodiments, R² is methyl. In certain embodiments, R² is ethyl.

In some examples, the compound of Formula (I) is

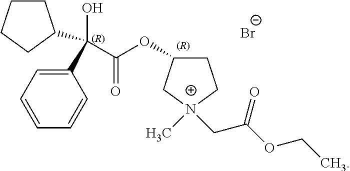

In some examples, the compound of Formula (I) is

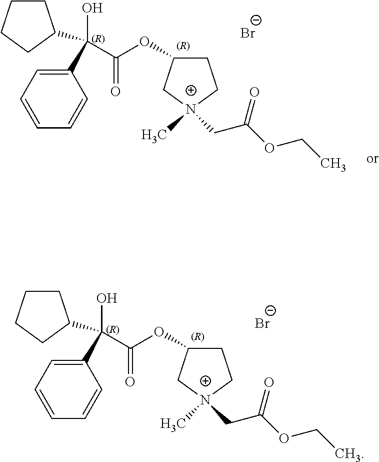

In some examples, the compound of Formula (I) is a threo mixture of:

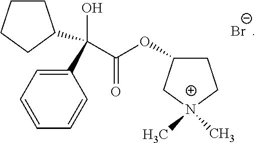

In some examples, the compound of Formula (I) is a threo mixture of:

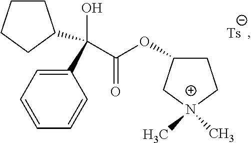

wherein Ts⁽⁻⁾ indicates a tosylate ion. In certain embodiments, the compound is a monohydrate.

In some examples, the compound is a racemic mixture of (R)-3-((S)-2-cyclopentyl-2-hydroxy-2-phenylacetoxy)-1,1-dimethylpyrrolidinium 4-methylbenzenesulfonate and (S)-3-((R)-2-cyclopentyl-2-hydroxy-2-phenylacetoxy)-1,1-dimethylpyrrolidinium 4-methylbenzenesulfonate. In certain embodiments, some examples, the compound is a racemic mixture of (R)-3-((S)-2-cyclopentyl-2-hydroxy-2-phenylacetoxy)-1,1-dimethylpyrrolidinium bromide and (S)-3-((R)-2-cyclopentyl-2-hydroxy-2-phenylacetoxy)-1,1-dimethylpyrrolidinium bromide.

In some examples, set forth herein is a composition including a mixture of compounds having following structures (Ia) and (Ib):

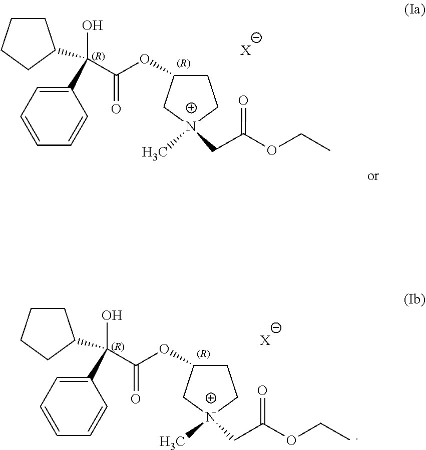

In some embodiments, including any of the foregoing, the anticholinergic compound has a structure according to Formula (II):

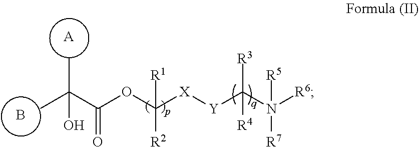

or a pharmaceutically acceptable salt thereof. Compounds of Formula (II) can be made, formulated, and administered according to U.S. Provisional Application No. 62/942,677 filed Dec. 2, 2019, the contents of which are hereby incorporated by reference in their entirety.

In Formula (II), ring A is selected from cycloalkyl, aryl, heterocycloalkyl, or heteroaryl. In some embodiments, ring A is optionally bridged to ring B. In Formula (II), ring B is selected from cycloalkyl, aryl, heterocycloalkyl, or heteroaryl. In some embodiments, ring B is optionally bridged to ring A. In Formula (II), ring A and ring B are each, independently in each instance, optionally substituted with 1-5 substituents selected from C₁₋₃alkyl, C₁₋₃alkoxyl, carbonyl, cyano, halo, hydroxyl, —NO₂, —NO₃, —SO₂, —SO₃, or —PO₄. In certain embodiments, X is O and Y is C═O. In certain other embodiments, X is C═O and Y is O. R¹ and R² are each, independently in each instance, selected from H, C₁₋₃alkyl, C₁₋₃alkoxyl, carbonyl, cyano, halo, hydroxyl, —NO₂, —NO₃, —SO₂, —SO₃, or —PO₄. If R¹ is carbonyl, then R² is not carbonyl. If R² is carbonyl, then R¹ is not carbonyl. If R³ is carbonyl, then R⁴ is not carbonyl. If R⁴ is carbonyl, then R³ is not carbonyl; R³ and R⁴ are each, independently in each instance, selected from H, C₁₋₃alkyl, C₁₋₃alkoxyl, carbonyl, cyano, halo, hydroxyl, —NO₂, —NO₃, —SO₂, —SO₃, or —PO₄. R⁵ is C₁₋₃alkyl or absent. R⁶ is C₁₋₃alkyl or bonded with R⁷ to form a five-membered heterocycloalkyl ring. and R⁷ is H, C₁₋₃alkyl, or bonded with R⁶ to form a five-membered heterocycloalkyl ring. In Formula (I), R¹, R², R³, R⁴, R⁵, R⁶, and R⁷ are unsubstituted. Subscript p is 1 or 2. Subscript q is 1, 2, or 3. When R⁵ is absent, the compound is preferably in the form of a pharmaceutically acceptable salt with a pharmaceutically acceptable counterion.

In some embodiments, including any of the foregoing, the compounds having the structure of Formula (II), are selected from Formula (Ha) or Formula (IIb):

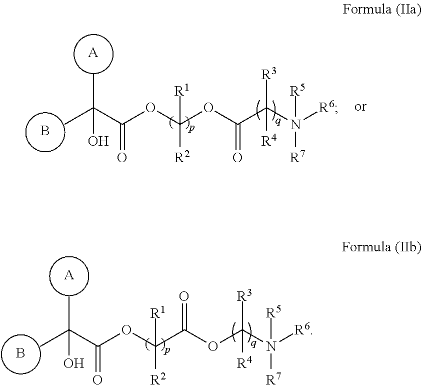

In some embodiments, including any of the foregoing, Ring A and Ring B are each, independently in each instance, either a five- or six-membered cycloalkyl, aryl, heterocycloalkyl, and heteroaryl. In certain embodiments, Ring A is five-membered cycloalkyl, aryl, heterocycloalkyl, and heteroaryl. In certain other embodiments, Ring A is six-membered cycloalkyl, aryl, heterocycloalkyl, and heteroaryl. In certain embodiments, Ring B is five-membered cycloalkyl, aryl, heterocycloalkyl, and heteroaryl. In certain other embodiments, Ring B is six-membered cycloalkyl, aryl, heterocycloalkyl, and heteroaryl. In some embodiments, Ring A is a five-membered cycloalkyl. In some other embodiments, Ring A is a five-membered aryl. In yet other embodiments, Ring A is a five-membered heterocycloalkyl. In still other embodiments, Ring A is a five-membered heteroaryl. In some embodiments, Ring A is a six-membered cycloalkyl. In some other embodiments, Ring A is a six-membered aryl. In yet other embodiments, Ring A is a six-membered heterocycloalkyl. In still other embodiments, Ring A is a six-membered heteroaryl. In some embodiments, Ring B is a five-membered cycloalkyl. In some other embodiments, Ring B is a five-membered aryl. In yet other embodiments, Ring B is a five-membered heterocycloalkyl. In still other embodiments, Ring B is a five-membered heteroaryl. In some embodiments, Ring B is a six-membered cycloalkyl. In some other embodiments, Ring B is a six-membered aryl. In yet other embodiments, Ring B is a six-membered heterocycloalkyl. In still other embodiments, Ring B is a six-membered heteroaryl.

In some embodiments, including any of the foregoing, the compounds having the structure of Formula (II), are selected from Formula (IIc) or Formula (IId):

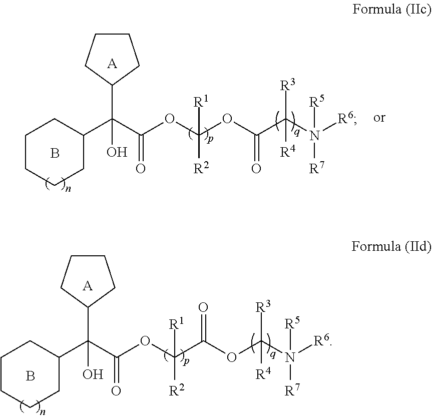

In Formula (IIc) or Formula (IId), subscript n is 0 or 1 such that when n is 0, ring B is a five-membered ring.

In some embodiments, including any of the foregoing, Ring A and Ring B are unsubstituted. In some embodiments, including any of the foregoing, Ring A is unsubstituted and Ring B is substituted. In some embodiments, including any of the foregoing, Ring A is substituted and Ring B is unsubstituted.

In some embodiments, including any of the foregoing, Ring A and Ring B are not bridged.

In some embodiments, including any of the foregoing, subscript p is 1 or 2. In some embodiments, subscript p is 1. In some other embodiments, subscript p is 2.

In some embodiments, including any of the foregoing, subscript q is 1 or 2. In some embodiments, subscript q is 1. In some other embodiments, subscript q is 2.

In some embodiments, including any of the foregoing, the compounds having the structure of Formula (II), are selected from Formula (IIe) or Formula (IIf):

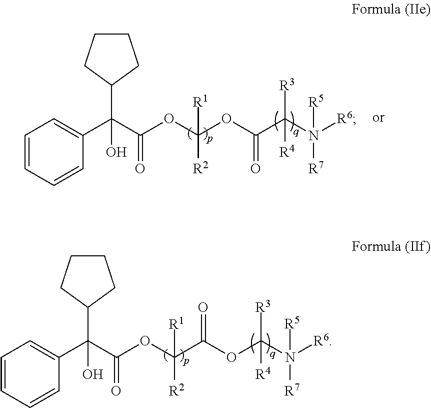

In some embodiments, including any of the foregoing, the compounds having the structure of Formula (II), are selected from Formula (IIg) or Formula (IIh):

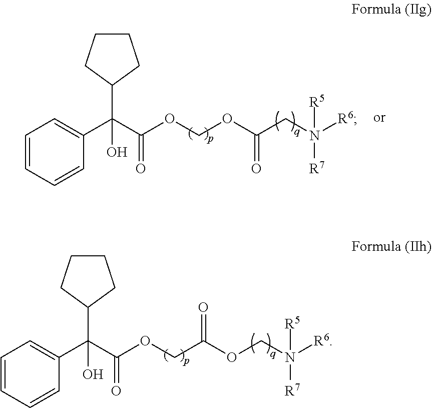

In some embodiments, including any of the foregoing, the compounds having the structure of Formula (II), are selected from Formula (IIi), Formula (IIj), Formula (IIk), Formula (IIl), Formula (IIm) or Formula (IIn):

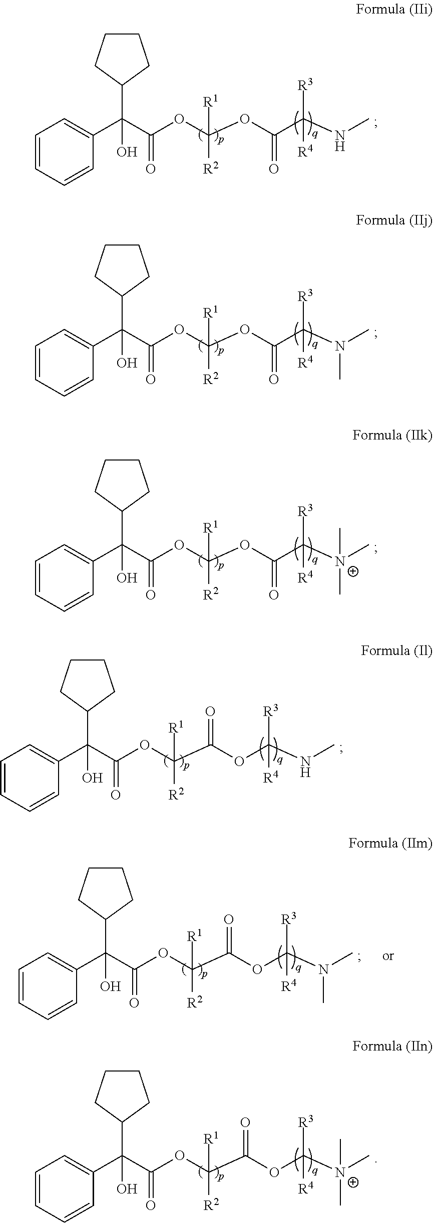

In some embodiments, including any of the foregoing, the compounds having the structure of Formula (II) are selected from Formula (IIo), Formula (IIp), Formula (IIq), Formula (IIr), Formula (IIs), Formula (IIt), Formula (IIu), or Formula (IIv):

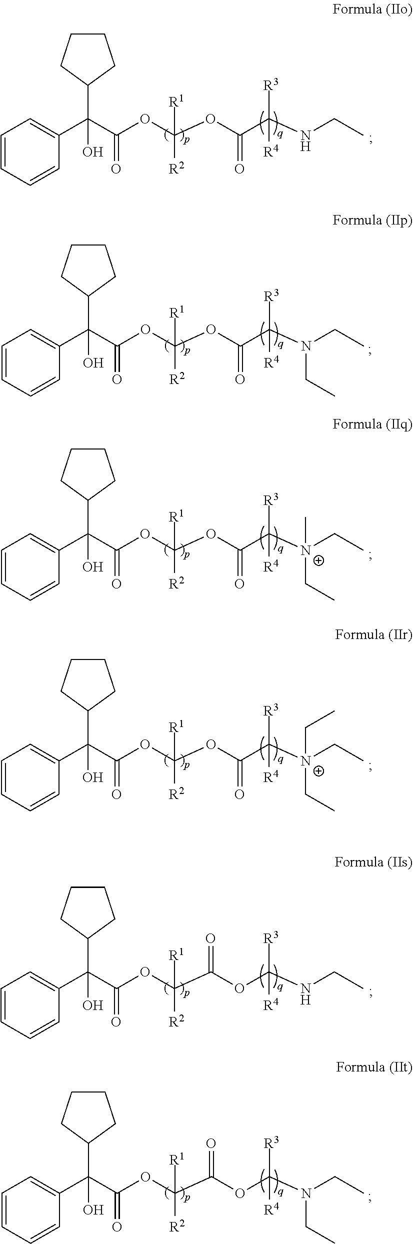

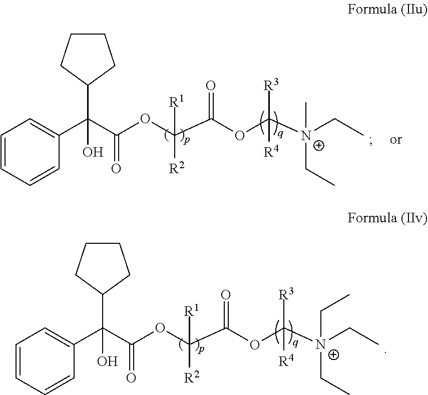

In some embodiments, including any of the foregoing, the compounds having the structure of Formula (II) are selected from Formula (IIw), Formula (IIx), Formula (IIy), or Formula (IIz):

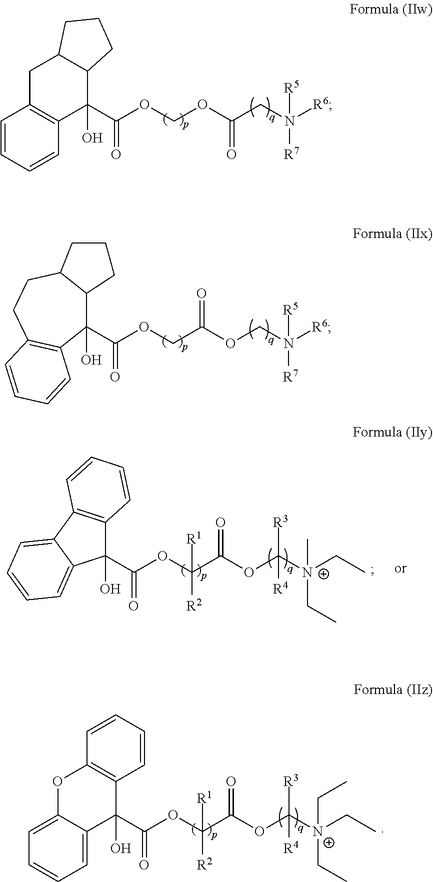

In some embodiments, including any of the foregoing, the compounds having the structure of Formula (II) are selected from:

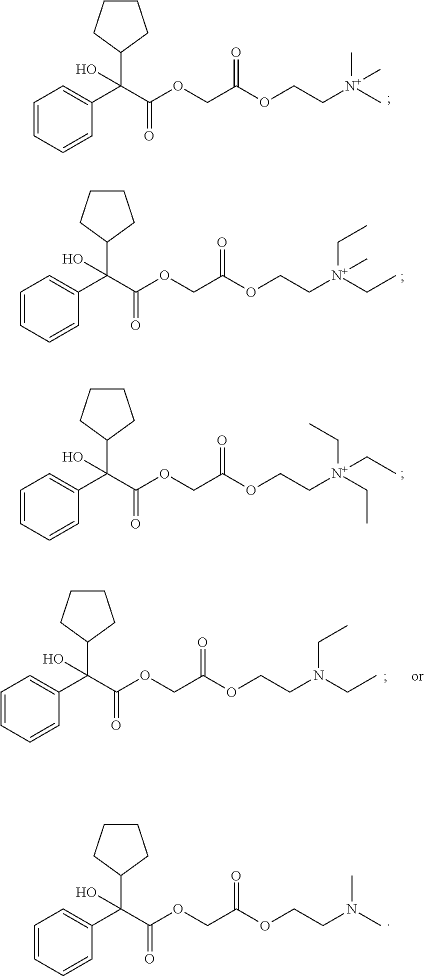 [SMILES: C[N+](C)(C)CCOC(=O)COC(=O)C(O)(c1ccccc1)C1CCCC1 ; CC[N+](C)(CC)CCOC(=O)COC(=O)C(O)(c1ccccc1)C1CCCC1 ; CC[N+](CC)(CC)CCOC(=O)COC(=O)C(O)(c1ccccc1)C1CCCC1 ; CCN(CC)CCOC(=O)COC(=O)C(O)(c1ccccc1)C1CCCC1 ; CN(C)CCOC(=O)COC(=O)C(O)(c1ccccc1)C1CCCC1]

In some embodiments, including any of the foregoing, the compounds having the structure of Formula (II) are selected from:

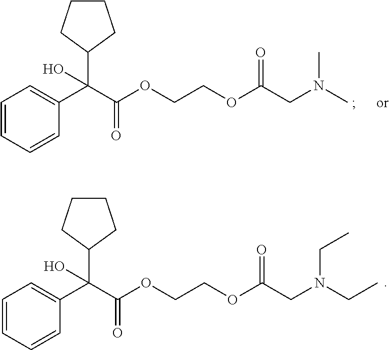 [SMILES: CN(C)CC(=O)OCCOC(=O)C(O)(c1ccccc1)C1CCCC1 ; CCN(CC)CC(=O)OCCOC(=O)C(O)(c1ccccc1)C1CCCC1]

In certain embodiments, the compound is stereochemically pure, i.e. in a diastereomeric excess. In certain embodiments, the stereomerically pure stereoisomer of the compound is present in an enantiomeric excess of at least 80%. In certain embodiments, the stereomerically pure stereoisomer of the compound is present in an enantiomeric excess of at least 85%. In certain embodiments, the stereomerically pure stereoisomer of the compound is present in an enantiomeric excess of at least 90%. In certain embodiments, the stereomerically pure stereoisomer of a compound of the compound is present in an enantiomeric excess of at least 95%. In certain embodiments, the stereomerically pure stereoisomer of a compound of the compound is present in an enantiomeric excess of at least 99%. In certain embodiments, the stereomerically pure stereoisomer of the compound is present in a diastereomeric excess of at least 80%. In certain embodiments, the stereomerically pure stereoisomer of the compound is present in a diastereomeric excess of at least 85%. In certain embodiments, the stereomerically pure stereoisomer of the compound is present in a diastereomeric excess of at least 90%. In certain embodiments, the stereomerically pure stereoisomer of the compound is present in a diastereomeric excess of at least 95%. In certain embodiments, the stereomerically pure stereoisomer of the compound is present in a diastereomeric excess of at least 99%.

In some examples, the compounds are formulated with a pharmaceutically acceptable excipient, diluent, or salt.

In some examples, set forth herein is a pharmaceutical composition, include a compound prepared by a method set forth herein. In some examples, the composition is formulated as a topical.

In some embodiments, the methods provided herein may further comprise administration of one or more additional agents, for instance to treat hyperhidrosis. Illustrative additional agents include any of those described in this disclosure or known in the art for the treatment of hyperhidrosis. The additional agent(s) may be administered in the same pharmaceutical composition as the agent recited in the methods provided herein, or in a different pharmaceutical composition, according to the judgment of those of skill in the art.

In some embodiments, the methods provided herein comprises administering a compound in combination with another agent or procedure. In some aspects, the other agent or procedure is selected from an anticholinergic agent, a metal salt, and a toxin.

In some embodiments, topical administration of an agent is combined with systemic administration of the same agent, or a different agent. In some aspects, topical administration of a glycopyrronium compound is combined with systemic administration of the glycopyrronium compound.

The compounds described herein can be administered alone or together with one or more additional therapeutic agents. The one or more additional therapeutic agents can be administered just prior to, concurrent with, or shortly after the administration of the compounds described herein. The present disclosure also includes pharmaceutical compositions comprising any of the compounds described herein in combination with one or more additional therapeutic agents, and methods of treatment comprising administering such combinations to subjects in need thereof.

The present disclosure includes pharmaceutical compositions of the compounds described herein, e.g., compositions comprising a compound described herein, a salt, stereoisomer, mixture of stereoisomers, polymorph thereof, and a pharmaceutically acceptable carrier, diluent, and/or excipient. Examples of suitable carriers, diluents and excipients include, but are not limited to: buffers for maintenance of proper composition pH (e.g., citrate buffers, succinate buffers, acetate buffers, phosphate buffers, lactate buffers, oxalate buffers and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, antioxidants, monohydric alcohols, such as ethanol, and polyhydric alcohols, such as glycols.

The compounds or compositions described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference in its entirety for all purposes.

Pharmaceutical compositions containing the compounds or compositions of the invention may be in any form suitable for the intended method of administration, including, for example, a solution, a suspension, or an emulsion. In some examples, the compositions set forth herein are suitable for topical application. In some examples, liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like.

The compounds can also be administered as prodrugs, where the prodrug undergoes transformation in the treated subject to a form which is therapeutically effective. Additional methods of administration are known in the art.

Compositions of the invention may be formulated to act at the epidermal layer, the dermal layer, or more deeply, i.e., greater than 500 μM deep, in tissue at, adjacent, or near sweat glands.

In certain embodiments, the pharmaceutical composition is in a topical applicator comprising about 1-15000 mg of glycopyrronium compound in an alcohol:water solution and with a pH buffering agent. In certain embodiments, the pharmaceutical composition is in a bottle comprising about 1-2500 mg of glycopyrronium compound in an alcohol:water solution and with a pH buffering agent. In certain embodiments, the pharmaceutical composition is in a topical applicator comprising about 1-1000 mg of glycopyrronium compound in an alcohol:water solution and with a pH buffering agent. In certain embodiments, the pharmaceutical composition is in a topical applicator comprising about 1-500 mg of glycopyrronium compound in an alcohol:water solution and with a pH buffering agent. In an embodiment, the glycopyrronium compound is present at a concentration of about 0.25-20% (w/w). In some embodiments, the glycopyrronium compound is present at a concentration of about 1, 2, 3, or 4% (w/w). In some embodiments, the glycopyrronium compound is present at a concentration of about 3% (w/w). In an embodiment, the topical composition comprises about 0.2 to about 1.0 g of the glycopyrronium compound. In an embodiment, the topical composition comprises about 540 mg of the glycopyrronium compound. In an embodiment, the alcohol:water ratio of the topical composition is selected over the range of 50:50 to 70:30, preferably over the range of 53:47 to 58:42. In an embodiment, the alcohol:water ratio of the topical composition is selected over the range of 45:55 to 70:30, for instance about 48% ethanol. In an embodiment, the buffering agent is about 0.2 to 0.5% of the topical composition. In an embodiment, the buffering agent of the topical composition is citric acid/sodium citrate. In an embodiment, the pH of the topical composition is selected over the range of 4.0 to 5.0. In an embodiment, the pH of the composition is about 4.5. In certain embodiments, the pharmaceutical composition comprises threo glycopyrronium tosylate monohydrate, citric acid, sodium citrate, ethanol, and water at a pH of about 4.5.

In certain embodiments, the pharmaceutical composition comprises a racemic mixture of (R)-3-((S)-2-cyclopentyl-2-hydroxy-2-phenylacetoxy)-1,1-dimethylpyrrolidinium 4-methylbenzenesulfonate and (S)-3-((R)-2-cyclopentyl-2-hydroxy-2-phenylacetoxy)-1,1-dimethylpyrrolidinium 4-methylbenzenesulfonate, about 0.15% by weight anhydrous citric acid, about 0.06% sodium citrate dihydrate by weight, between about 57 to about 59.5% by weight of dehydrated ethanol, and the balance as water. In certain embodiments, the pharmaceutical composition comprises a racemic mixture of (R)-3-((S)-2-cyclopentyl-2-hydroxy-2-phenylacetoxy)-1,1-dimethylpyrrolidinium 4-methylbenzenesulfonate and (S)-3-((R)-2-cyclopentyl-2-hydroxy-2-phenylacetoxy)-1,1-dimethylpyrrolidinium 4-methylbenzenesulfonate, about 0.15% by weight anhydrous citric acid, about 0.06% sodium citrate dihydrate by weight, between about 45 to 50%, for instance about 48%, by weight of dehydrated ethanol, and the balance as water.

In certain embodiments, the pharmaceutical composition for a 25 ml bottle comprises about 0.1 g to about 10 g, about 0.1 g to about 5 g, about 0.1 g to about 3 g, about 0.1 g to about 2 g, about 0.1 g to about 1 g, about 120 mg, about 1 g, about 2 g, or about 2.5 g, or about 3 g of a racemic mixture of (R)-3-((S)-2-cyclopentyl-2-hydroxy-2-phenylacetoxy)-1,1-dimethylpyrrolidinium 4-methylbenzenesulfonate and (S)-3-((R)-2-cyclopentyl-2-hydroxy-2-phenylacetoxy)-1,1-dimethylpyrrolidinium 4-methylbenzenesulfonate, about 0.15% by weight anhydrous citric acid, about 0.06% sodium citrate dihydrate by weight, between about 57 to about 59.5% by weight of dehydrated ethanol, and the balance as water. In certain embodiments, the dehydrated ethanol percentage is about 45 to about 50%, for instance about 48%. For a different size bottle, the amounts would vary accordingly.

In certain embodiments, the applicator maintains stability of the anticholinergic compound composition. In certain embodiments, the applicator provides weight loss of the composition comprising the anticholinergic compound in a range of less than 5%, less than 1%, for instance about 0.01 to 0.03%, by weight after two weeks at a temperature of about 25° C. and a relative humidity of about 60% with the applicator in a shelf-life configuration. In certain embodiments, the applicator provides weight loss of the composition in a range of less than 5%, less than 1%, for instance about 0.04 to 0.06%, by weight after one month at a temperature of about 25° C. and a relative humidity of about 60% with the applicator in a shelf-life configuration. In certain embodiments, the applicator provides weight loss of the composition in a range of less than 5%, less than 1%, for instance about 0.1 to 0.25% by weight after two weeks at a temperature of about 40° C. and a relative humidity of about 75% with the applicator in a shelf-life configuration. In certain embodiments, the applicator provides weight loss of the composition in a range of less than 5%, less than 1%, for instance about 0.25 to 0.6%, by weight after one month at a temperature of about 40° C. and a relative humidity of about 75% with the applicator in a shelf-life configuration. In certain embodiments, the applicator provides weight loss of the composition in a range of less than 5%, less than 1%, for instance about 0.0 to 0.3%, by weight after one week at ambient temperature. In certain embodiments, the applicator provides weight loss of the composition in a range of less than 5%, less than 1%, for instance about 0.0 to 0.35%, by weight after two weeks at ambient temperature. In certain embodiments, the applicator provides weight loss of the composition in a range of less than 5%, less than 1%, for instance about 0.0 to 0.05%, by weight after two weeks at a temperature of about 25° C. and a relative humidity of about 60% with the applicator in an in-use configuration. In certain embodiments, the applicator provides weight loss of the composition in a range of less than 5%, less than 1%, for instance about 0.05 to 0.15%, by weight after two weeks at a temperature of 40° C. and a relative humidity of about 75% with the applicator in an in-use configuration. In certain embodiments, the applicator provides weight loss of the composition in a range of less than 30%, for instance about 15 to 25%, by weight after two weeks at a temperature of 60° C. with the applicator in an in-use configuration.

### D. Kits

Also provided herein are kits for use in the methods provided herein. In some embodiments, the kit comprises an applicator containing a compound or a pharmaceutical composition comprising a compound. In certain embodiments, the kit comprises two or more components of the applicator. In certain embodiments, the kit comprises two or more components of the applicator, and instructions for their use. The kit can further comprise instructions, for instance for administration, occlusion, and/or monitoring. The kit can further comprise materials for an assessment, e.g. an HDSS assessment or an ASDD assessment modified as described herein, or both.

In some embodiments, the kit further comprises packaging. The kit may comprise a bottle, a cap, and the roll-on assembly as described above with respect to various embodiments. In some aspects, this packaging includes a container suitable for holding a pharmaceutical composition. The container can be made of any suitable material. Suitable materials include, for example, glass, plastic paper, laminates, and the like.

### EXAMPLES

Unless otherwise stated, chemical reagents were purchased from commercially available sources.

Reagents used herein are available from commercial vendors and were purchased from commercially available sources unless specified herein otherwise or unless the preparation of the reagent(s) is/are described herein.

### Example 1

Sample applicators were filled with a 70% isopropyl alcohol solution and tested. Testing included inversion, squeeze application (without an over cap), and quick-evaporation. Tubes/tubals were sealed with a clip. Bottles and tube/tubals were closed with threaded, snap-on, or press-on rollerball closures with over caps.

All designs were able to contain the liquid within the bottle or tube/tubal format. Samples were then evaluated for leakage immediately upon fill and thereafter. The bottles/tubes were then inverted and capped for 6 to 12 hours to determine further leakage and liquid evaporations.

Of approximately 30 applicators tested, only four were found not to leak on inversion and not to leak on squeezing. The applicator includes a bottle with a design that allows the applicator to present a 30-day to 60-day supply in an ergonomically pleasing bottle, i.e. a bottle that is not too small to hold. The shape permits use with a wider roller for convenient, comfortable application, for instance to axillary skin. The cap seals the bottle for shelf-life stability. The roller assembly is easily installed by the patient, caregiver, or pharmacists at time of use. The two configurations (bottle with cap for shelf life storage and roll-on assembly on bottle for in use) ensure good shelf life stability. The applicator itself is described in detail in the sections above.

The present example provides compound stability measured in an applicator set forth herein. Stability data is set forth in the following tables.

TABLE 1
Shelf-Life Configuration
Upright orientation (weight loss relative to fill of 18 g)

| | Range of weight loss—Set A | | Range of weight loss—Set B | |
|---|---|---|---|---|
| | 25° C./60% RH (n = 5) | | 40° C./75% RH (n = 5) | |
| | | Weight | | Weight |
| Time | Weight loss (g) | loss (%) | Weight loss (g) | loss (%) |
| T0 | NA | NA | NA | NA |
| 2 | 0.00473 ± 0.00492 | 0.026 ± 0.027 | 0.02526 ± 0.03819 | 0.140 ± 0.202 |
| weeks | | | | |
| 1 | 0.00921 ± 0.00955 | 0.051 ± 0.053 | 0.05470 ± 0.09037 | 0.304 ± 0.502 |
| month | | | | |

Here, a shelf life configuration is one in which the bottle holding the compound is capped. In Table 1, the results shown indicate, for conditions in Set A, a range of weight loss may be expected to be from about 0.01 to 0.03% by weight after two weeks, and of from about 0.04 to 0.06% by weight after one month. For conditions in Set B, a range of weight loss may be expected to be from about 0.1 to 0.25% by weight after two weeks, and from about 0.25 to 0.6% by weight after one month. For different shelf life tests, the amount of weight loss may be proportional to the amounts shown here.

TABLE 2
In-Use Configuration
Upright orientation (weight loss relative to fill of 18 g)

| | Range of Weight Loss with Cap |
|---|---|
| | Ambient Temperature (n = 5) |
| Time | Weight loss (g) |
| 1 week | 0.00113-0.03011 g (0.0-0.2%) |
| 2 weeks | 0.00206-0.04303 g (0.0-0.2%) |

Here, an in use configuration is one in which the bottle holding the compound is contained within the sleeve, and the sleeve is capped. In Table 2, for the conditions shown, the results shown indicate a range of weight loss may be expected to be from about 0 to 0.3% after one week, and about the same after two weeks. For different in-use tests, the amount of weight loss may be proportional to the amounts shown here.

TABLE 3
In-Use Configuration
Horizontal orientation (weight loss relative to fill of 45 g)

| | Range of | Range of | Range of |
|---|---|---|---|
| | weight loss | weight loss | weight loss |
| | 25° C./60% | 40° C./75% | 60° C. |
| Time | RH (n = 6) | RH (n = 6) | (n = 6) |
| 2 weeks | 0.003-0.1846 g | 0.0368-0.5336 g | 9.1783-10.2979 g |
| | (0.01-0.41%) | (0.063-0.14%) | (20.4-22.9%) |

Here, an in use configuration is one in which the bottle holding the compound is contained within the sleeve, and the sleeve is capped. In Table 3, with the bottle in the horizontal orientation as indicated, data shows loss for evaporation and leakage together. For the conditions shown, the results shown indicate a range of weight loss may be expected to be from about about 0 to 0.5% after two weeks at the recommended storage conditions (shown in the second column in Table 3), from about 0.05 to 0.15% after two weeks at the accelerated conditions (shown in the third column in Table 3), and about 15 to 25% after two weeks under stress conditions (shown in the fourth or far right column of Table 3).

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. An applicator for topical administration of an anticholinergic compound, or a pharmaceutically acceptable salt or composition thereof, to a subject in need thereof, the applicator comprising:
a. a bottle containing the compound, the bottle comprising bottle connector structure at an upper portion thereof; and
b. a sleeve comprising an applicator member, at a top portion of the sleeve, for administering the compound, and sleeve connector structure to mate with the bottle connector structure to provide communication between the bottle and the applicator member to enable administration of the compound,
wherein the bottle connector structure and the sleeve connector structure comprise structure selected from the group consisting of respective screw threads, respective snap fit structures, and respective press fit structures to mate the bottle and the sleeve when the bottle is screwed into the sleeve.

2. (canceled)

3. (canceled)

4. (canceled)

5. (canceled)

6. The applicator of claim 1 wherein mating of the bottle connector structure and the sleeve connector structure provides a seal to reduce leakage and/or evaporation of a medium for the anticholinergic compound, pharmaceutically acceptable salt or composition thereof.

7. (canceled)

8. The applicator of claim 1 wherein the applicator comprises one or a plurality of rollers disposed at the top portion of the sleeve, the roller or rollers selected from the group consisting of spheroids and ellipsoids each rotatable about a major axis, the roller or rollers sized to administer a desired amount of the compound.

9. (canceled)

10. The applicator of claim 8 wherein one or more of the bottle, the sleeve, or the roller or rollers comprises material selected from the group consisting of polypropylene (including high-density polypropylene), polyethylene (including high-density polyethylene), polytetrafluoroethylene (PTFE), cyclic co-polymer, nylon, and polyethylene terephthalate (PET).

11. (canceled)

12. (canceled)

13. (canceled)

14. The applicator of claim 1 wherein the bottle comprises a reservoir to hold the compound, and a neck containing the bottle connector structure at an upper portion of the bottle, the neck sized to fit within the sleeve.

15. The applicator of claim 14 wherein the reservoir comprises at least a portion of translucent material to make visible an amount of the compound present.

16. (canceled)

17. (canceled)

18. The applicator of claim 14 wherein an upper portion of the reservoir is press fit or snap fit into the sleeve.

19. (canceled)

20. (canceled)

21. The applicator of claim 1 wherein the bottle comprises a bottle cap that attaches to the bottle via the bottle connector structure, wherein removal of the bottle cap enables the sleeve connector structure to mate with the bottle connector structure to reduce leakage and/or evaporation.

22. The applicator of claim 21 wherein the bottle cap attaches to the bottle via an attachment structure respectively in the bottle cap and the bottle connector structure, the attachment structure selected from the group consisting of respective screw threads, respective snap fit structures, and respective press fit structures.

23. (canceled)

24. (canceled)

25. (canceled)

26. (canceled)

27. (canceled)

28. The applicator of claim 21 wherein the sleeve further comprises a sleeve cap connector structure at the top portion of the sleeve below the roller, and a sleeve cap comprising a cap ring extending downwardly from inside the sleeve cap to contact the roller and compress the roller, the sleeve further comprising a ring structure which contacts the roller to urge the roller downwardly from the cap, so as to reduce leakage and/or evaporation.

29. The applicator of claim 28 further comprising a compressible structure attached to the ring structure, the compressible structure compressing when contacting the roller to urge the roller downwardly from the cap so as to reduce leakage and/or evaporation.

30. (canceled)

31. The applicator of claim 28 wherein the sleeve cap comprises a covering portion extending downwardly from a top of the sleeve cap to cover or compress the roller to reduce leakage and/or evaporation.

32. (canceled)

33. The applicator of claim 28 wherein one or more of the sleeve, the bottle cap, or the sleeve cap has one or more of a textured surface, finger grips, or variable diameter to facilitate gripping of the or more of the sleeve, the bottle cap, or sleeve cap.

34. (canceled)

35. (canceled)

36. (canceled)

37. (canceled)

38. (canceled)

39. The applicator of claim 1 wherein weight loss of the anticholinergic compound composition is in a range of about 0.0 to 0.35% by weight after two weeks at ambient temperature.

40. (canceled)

41. (canceled)

42. (canceled)

43. The applicator of claim 1 wherein the composition comprises at least 48% ethanol.

44. (canceled)

45. The applicator of claim 28 further comprising structure selected from the group consisting of respective screw threads, snap fit structures, and press fit structures in the sleeve cap and the sleeve cap connector structure to attach the sleeve cap to the sleeve.

46. The applicator of claim 28 wherein one or more of the sleeve cap or the bottle cap engages respectively with the sleeve or bottle sufficiently tightly to constitute a child resistant structure.

47. (canceled)

48. (canceled)

49. (canceled)

50. (canceled)

51. (canceled)

52. (canceled)

53. (canceled)

54. (canceled)

55. The applicator of claim 1 wherein the anticholinergic compound is a compound of Formula (I): 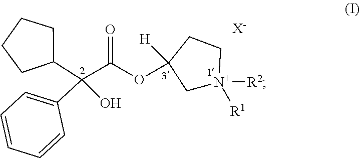
wherein:
R¹ and R² are each, independently in each instance, selected from alkyl and alkyl substituted with alkoxycarbonyl;
X⁻ is an anion;
or a pharmaceutically acceptable salt or solvate thereof.

56. The applicator of claim 55 wherein the stereochemical configuration about the carbon atom is a racemic threo mixture of 2/3′ R/S and 2/3′ S/R.

57. The applicator of claim 55 wherein the stereochemical configuration about the carbon atom indicated by 2 is R and the stereochemical configuration about carbon atom indicated by 3′ is R.

58. The applicator of claim 1 wherein the anticholinergic compound is a compound selected from the group consisting of a glycopyrronium compound, threo glycopyrronium bromide, threo glycopyrronium tosylate, threo glycopyrronium tosylate monohydrate, a sofpironium compound, sofpironium bromide, propantheline, oxybutynin, methantheline, and benztropine.

59. (canceled)

60. (canceled)

61. (canceled)

62. (canceled)

63. (canceled)

64. The applicator of claim 1 wherein the anticholinergic compound is a compound selected from the group consisting of:

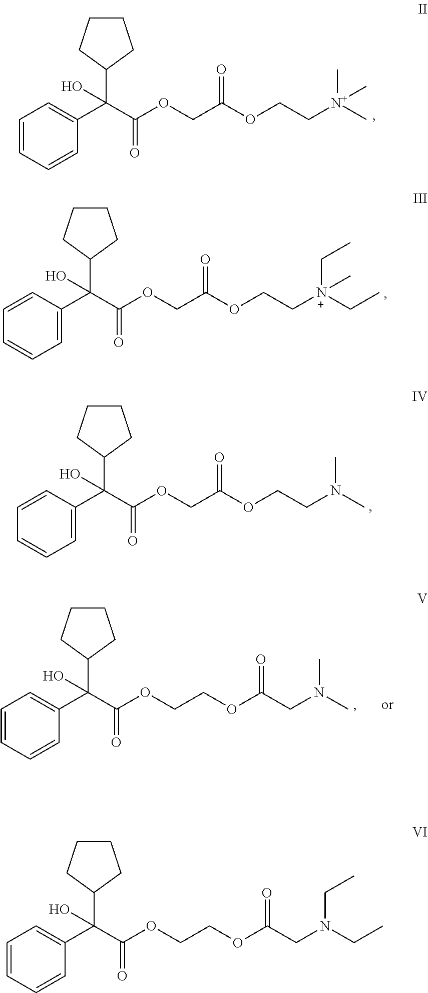 [SMILES: C[N+](C)(C)CCOC(=O)COC(=O)C(O)(c1ccccc1)C1CCCC1 ; CC[N+](C)(CC)CCOC(=O)COC(=O)C(O)(c1ccccc1)C1CCCC1 ; CN(C)CCOC(=O)COC(=O)C(O)(c1ccccc1)C1CCCC1 ; CN(C)CC(=O)OCCOC(=O)C(O)(c1ccccc1)C1CCCC1 ; CCN(CC)CC(=O)OCCOC(=O)C(O)(c1ccccc1)C1CCCC1]

65. The applicator of claim 1 wherein the anticholinergic compound is in a pharmaceutical composition comprising the compound and a pharmaceutically acceptable carrier, excipient, or diluent.

66. The applicator of claim 1 wherein the pharmaceutical composition is formulated as a topical composition.

67. The applicator of claim 1 wherein the dose of the compound is from about 0.1 to about 0.8 mg.

68. (canceled)

69. A method of treating a condition in a subject in need thereof, comprising the step of administering to the subject an amount of a compound, or a pharmaceutically acceptable salt or a pharmaceutical composition thereof, with an applicator of claim 1.

70. (canceled)

71. The method of claim 69 wherein the subject is a mammal and the condition is selected from the group consisting of hyperhidrosis and axillary hyperhidrosis.

72. (canceled)

73. (canceled)

74. (canceled)
